(19) United States
(12) Patent Application Publication
(10) Pub. No.: US 2026/0240891 A1
(43) Pub. Date: Aug. 20, 2026

(54) FITUSIRAN FOR THE TREATMENT OF HEMOPHILIA A AND B IN PEDIATRIC PATIENTS

(71) Applicant: Genzyme Corporation, Cambridge, MA (US)

(72) Inventors: Shauna ANDERSSON, Lexington, MA (US); Pratik R BHAGUNDE, New Providence, NJ (US); Sajida IQBAL, Birmingham (GB); Pranav PATEL, Warrington, PA (US); Stacey POLOSKEY, Sudbury, MA (US); Fadi SHAMMAS, Montreal (CA)

(21) Appl. No.: 18/872,107

(22) PCT Filed: Jun. 8, 2023
(86) PCT No.: PCT/US2023/068137

§ 371 Date: Dec. 5, 2024

Related U.S. Application Data
(60) Provisional application No. 63/350,382, filed on Jun. 8, 2022.
(60) Provisional application No. 63/381,499, filed on Oct. 28, 2022.

(51) Int. Cl. A61K 31/713; A61K 9/00; A61K 31/7115; A61K 31/712; A61K 31/7125; A61K 38/48; A61P 7/04

(52) U.S. Cl. CPC A61K 31/713; A61K 9/0019; A61K 31/7115; A61K 31/712; A61K 31/7125; A61K 38/4833; A61K 38/4846; A61P 7/04

(57) ABSTRACT

The present disclosure provides methods and compositions for treating pediatric patients with hemophilia A or B with or without inhibitors.

### CROSS-REFERENCE TO RELATED APPLICATIONS

The present application claims priority from U.S. Applications 63/350,382, filed Jun. 8, 2022, and 63/381,499, filed Oct. 28, 2022. The contents of the priority applications are incorporated by reference herein in their entirety.

### SEQUENCE LISTING

The instant application contains a Sequence Listing which has been submitted electronically in XML format and is hereby incorporated by reference in its entirety. Said XML copy, created on Jun. 2, 2023, is named 022548WO089.xml and is 8,045 bytes in size.

### BACKGROUND OF THE INVENTION

Hemophilia A and hemophilia B are X-linked recessive inherited bleeding disorders, characterized by deficiency of coagulation factor VIII (FVIII) or factor IX (FIX), leading to a profound defect of thrombin generation with impaired hemostasis and increased risk of bleeding. Factor replacement concentrates are the current standard of care for hemophilia patients without inhibitory antibodies (i.e., inhibitors) to FVIII or FIX but remain associated with high treatment burden due to the frequency of intravenous (IV) administration (2 to 3 times per week or more) to prophylactically maintain hemostasis. In addition, the development of inhibitors is a major complication in children treated with factor replacement. Approximately 30% of previously untreated patients with hemophilia A and 2% to 5% of previously untreated patients with hemophilia B develop inhibitory antibodies to FVIII and FIX, respectively (Gouw et al., N Engl J Med. (2013) 368(3): 231-9; and Puetz et al., Haemophilia. (2014) 20(1): 25-31).

Treatment of inhibitor patients may involve immune tolerance induction (ITI) therapy in an attempt to eradicate inhibitors and/or require the use of bypassing agents (BPA) such as activated prothrombin complex concentrates (aPCC) and recombinant activated factor VII (rFVIIa). However, failure rates of various ITI approaches are high, especially for hemophilia B patients, and later inhibitor recurrence following ITI may occur. Inhibitor patients using BPAs have shown higher annual bleeding rates and increased treatment burden over factor replacement therapies used by noninhibitor patients (Antunes et al., Haemophilia. (2014) 20(1): 65-72; Konkle et al., J Thromb Haemost. (2007) 5(9): 1904-13).

Another potential option for patients with hemophilia A and inhibitors to FVIII is recently approved emicizimab, a bispecific antibody that mimics the function of FVIII. While emicizimab has shown promise in this population, clinical events of thrombosis have been observed when emicizimab is used in combination with specific BPAs that require risk mitigation strategies and an accompanying black box warning.

For pediatric patients with hemophilia, the treatment of both inhibitor and noninhibitor populations is further complicated by challenges of venous access and caregiver burden. For noninhibitor pediatric patients, more frequent infusions may be required due to a shorter half-life of factor replacement therapies than in adults. In the case of inhibitor patients, prophylactic BPA therapy or ITI regimens may require daily or every other day infusions.

Thus, there remains a need for alternative treatments for pediatric patients having hemophilia A or B.

### SUMMARY OF THE INVENTION

The present disclosure provides methods of prophylactically treating hemophilia A or B with or without inhibitors with fitusiran to prevent or reduce the frequency of bleeding episodes in pediatric patients who are from 1 to less than 12 year(s) of age, and provides fitusiran for use in these methods.

In one aspect, the present disclosure provides a method of treating hemophilia A or B in a pediatric patient with or without inhibitors, comprising: (a) subcutaneously administering to the patient in need thereof fitusiran at a starting dose amount at a selected dosing frequency; (b) obtaining a measurement of an antithrombin (AT) level in the patient; and (c) performing one of the following steps: (i) if the AT level is 15-35%, repeating step (a), (ii) if the AT level is >35%, subcutaneously administering to the patient fitusiran at a higher dose amount at the selected dosing frequency or at the starting dose amount at a higher dosing frequency, or (iii) if the AT level is <15%, subcutaneously administering to the patient fitusiran at a lower dose amount at the selected dosing frequency or at the starting dose amount at a lower dosing frequency, optionally wherein step (c) (iii) is performed after a pause in fitusiran administration and after the AT level in the patient has returned to ≥15%, optionally ≥22%.

In some embodiments, the method reduces the frequency of bleeding episodes in the patients, e.g., reducing their annual bleed rates (ABR), annual spontaneous bleeding rates (AsBR), and/or annual joint bleeding rates (AjBR). The method may also reduce thrombotic risk in a pediatric patient receiving fitusiran for prophylactic treatment of hemophilia A or B with or without inhibitors.

In another aspect, the present disclosure provides a series of methods of treating hemophilia A or B in a pediatric patient with or without inhibitors using a pre-determined amount of fitusiran (e.g., 1.25, 2.5, 5, 7.5, 10, 20, 30, or 50 mg) at a pre-determined frequency (e.g., every two months, every eight weeks, every month, or every four weeks).

In another aspect, the present provides fitusiran formulations suitable for the methods herein. In some embodiments, the fitusiran formulation is an aqueous fitusiran composition comprising: about 12.5 mg/mL fitusiran, about 0.388 mg/mL NaH₂PO₄*H₂O, about 0.586 mg/mL Na₂HPO₄*7H₂O, and about 8.7 mg/mL NaCl, with a pH of about 7.0-7.1.

Also provided herein are fitusiran and articles of manufacture for use in the present treatment methods, use of fitusiran for the manufacture of a medicament for treating hemophilia A or B in a pediatric patient with or without inhibitors in the present methods, and a pharmaceutical composition comprising fitusiran for use in the present treatment methods.

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIG. 1 shows the expanded structural formula, chemical formula, and molecular mass of fitusiran (sodium form). This figure discloses SEQ ID NOs: 1 and 2, respectively, in order of appearance.

FIG. 2 is a diagram showing the study design described in Example 1. AT (Antithrombin) Follow up: antithrombin activity levels are monitored at monthly intervals in participants who do not continue fitusiran treatment until AT activity levels return to approximately 60% per the central laboratory, or per Investigator discretion in consultation with the study Medical Manager.

FIG. 3 is a flow chart depicting the fitusiran escalation/de-escalation scheme for patients in Cohort 1 (patients with a body weight of 22 kg to <45 kg). Participants will start/resume fitusiran at a dose of 10 mg every 4 weeks. AT: antithrombin activity; QM=once monthly; SS=steady state. *: within a 12-month period; **: start of dosing after de-escalation from higher dose to occur only after centrally measured AT activity levels ≥22%.

FIG. 4 is a flow chart depicting the fitusiran escalation/de-escalation scheme for patients in Cohort 2 (patients with a body weight of 8 kg to <22 kg). Participants will start fitusiran at a dose of 5 mg every 4 weeks. Abbreviations and symbols are the same as explained for FIG. 3 above.

FIG. 5 is a schematic depicting the pharmacokinetic/pharmacodynamic (PK/PD) model used to simulate the dynamics of plasma AT activity for patients treated with fitusiran. KA: absorption rate; F: bioavailability with dose effect on bioavailability; V2: liver central compartment volume; V₃ liver peripheral compartment volume; CL: clearance from liver central compartment; CL₂: liver intercompartmental clearance; Q: clearance into RISC compartment; RV: RISC volume; CL_{R}: clearance out of RISC compartment; Kᵢₙ: AT production rate; Kₒᵤₜ: AT elimination rate; Iₘₐₓ: maximum inhibition of AT production; IC₅₀: RISC concentration for 50% of maximum inhibition of AT production.

FIG. 6 is a table showing computer modeling data for the distribution of trough and peak simulated AT activity for pediatric cohorts for various dosing regimens. WT: bodyweight. Min.: minimum. Max.: maximum.

FIGS. 7A and 7B are diagrams showing the computationally predicted distribution of AT levels in Cohort 1 patients treated with a starting dose of 10 mg fitusiran once a month. FIG. 7A shows the de-escalation scheme for the predicted 26% of patients with AT values <15% after treatment with the 10 mg QM starting dose. FIG. 7B shows the escalation scheme for the predicted 10% of patients with AT values >35% after treatment with the 10 mg QM starting dose.

FIGS. 8A and 8B are diagrams showing the computationally predicted distribution of AT levels in Cohort 2 patients treated with a starting dose of 5 mg fitusiran once a month. FIG. 8A shows the de-escalation scheme for the predicted 21% of patients with AT values <15% after treatment with the 5 mg QM starting dose. FIG. 8B shows the escalation scheme for the predicted 19% of patients with AT values >35% after treatment with the 5 mg QM starting dose.

### DETAILED DESCRIPTION OF THE INVENTION

Fitusiran is a N-acetylgalactosamine (GalNAc) small interfering ribonucleic acid (siRNA) conjugate that reduces production of antithrombin (AT), leading to lower plasma AT activity levels. By reducing plasma AT, fitusiran is designed to improve thrombin generation and hemostasis in individuals with hemophilia, regardless of hemophilia type or presence of inhibitory antibodies to FVIII or FIX. Fitusiran is being developed for routine prophylaxis to prevent or reduce the frequency of bleeding episodes in patients with hemophilia A or B, including patients with inhibitory antibodies to FVIII or FIX.

A subcutaneous (SC) therapy that can effectively and safely prevent or reduce the frequency of bleeding episodes in patients with hemophilia A or B, including those with inhibitors, may reduce treatment burden, improve clinical outcomes and enhance quality of life, especially for the pediatric patient population.

The present disclosure provides a method that aims to maintain a favorable benefit-risk balance for pediatric patients with hemophilia A or B with or without inhibitors who are treated with fitusiran. Fitusiran is intended for routine prophylaxis to prevent or reduce the frequency of bleeding episodes in pediatric patients (i.e., 1 to <12 year(s) old), with hemophilia A or B, including patients with inhibitory antibodies (inhibitors). The present treatment methods carefully calibrate the therapy based on the patient's AT levels so as to minimize the risk of vascular thrombotic events that may result from low AT levels (e.g., AT levels <10%).

A hemophilia A or B patient with inhibitors refers to a patient who has developed alloantibodies to the factor he/she has previously received (e.g., factor VIII for hemophilia A patients or factor IX for hemophilia B patients). A hemophilia A or B patient with inhibitors may become refractory to replacement coagulation factor therapies. A patient without inhibitors refers to a patient who does not have such alloantibodies. The present treatment methods may be beneficial for hemophilia A patients with or without inhibitors, as well as for hemophilia B patients with or without inhibitors. As used herein, “hemophilia A or B with or without inhibitors” refers to hemophilia A with or without inhibitors, or hemophilia B with or without inhibitors. As used herein, a patient refers to a human pediatric patient that is 1 to less than 12 year(s) of age.

### I. Fitusiran Pharmaceutical Compositions

Hemophilia results in a profound defect in thrombin generation, and further, hemophilia severity is correlated with the inability to generate thrombin. Without being bound by theory, it is believed that fitusiran-mediated lowering of antithrombin (AT) levels will increase thrombin generation and thus improve hemostasis in patients with hemophilia. Antithrombin is encoded by the SERPINCI gene.

Fitusiran, whose structure is provided herein, is a synthetic, chemically modified double-stranded small interfering RNA (siRNA) oligonucleotide covalently linked to a tri-antennary N-acetyl-galactosamine (GalNAc) ligand targeting the AT3 mRNA in the liver, thereby suppressing the synthesis of antithrombin. The nucleosides in each strand of fitusiran are connected through either 3′-5′ phosphodiester or phosphorothioate linkages, thus forming the sugar-phosphate backbone of the oligonucleotide. The sense strand and the antisense strand of fitusiran contain 21 and 23 nucleotides, respectively. The 3′ end of the sense strand is conjugated to the GalNAc containing moiety (referred to as L96) through a phosphodiester linkage. The sense strand contains two consecutive phosphorothioate linkages at its 5′ end. The antisense strand contains four phosphorothioate linkages, two at the 3′ end and two at the 5′ end. The 21 nucleotides of the sense strand hybridize with the complementary 21 nucleotides of the antisense strand, thus forming 21 nucleotide base pairs and a two-base overhang at the 3′-end of the antisense strand. See also U.S. Pat. Nos. 9,127,274, 11,091,759, and WO 2019/014187.

The two nucleotide strands of fitusiran are shown below:

sense strand:
(SEQ ID NO: 1)
5′ Gf-ps-Gm-ps-Uf-Um-Af-Am-Cf-Am-Cf-Cf-Af-Um-Uf-
Um-Af-Cm-Uf-Um-Cf-Am-Af-L96 3′,
and
antisense strand:
(SEQ ID NO: 2)
5′ Um-ps-Uf-ps-Gm-Af-Am-Gf-Um-Af-Am-Af-Um-Gm-Gm-
Uf-Gm-Uf-Um-Af-Am-Cf-Cm-ps-Am-ps-Gm 3′,

wherein
Af=2′-fluoroadenosine (i.e., 2′-deoxy-2′-fluoroadenosine)
Cf=2′-fluorocytidine (i.e., 2′-deoxy-2′-fluorocytidine)
Gf=2′-fluoroguanosine (i.e., 2′-deoxy-2′-fluoroguanosine)
Uf=2′-fluorouridine (i.e., 2′-deoxy-2′-fluorouridine)
Am=2′-O-methyladenosine
Cm=2′-O-methylcytidine
Gm=2′-O-methylguanosine
Um=2′-O-methyluridine
“-” (hyphen)=3′-5′ phosphodiester linkage sodium salt
“-ps-”=3′-5′ phosphorothioate linkage sodium salt
   and wherein L96 has the following formula:

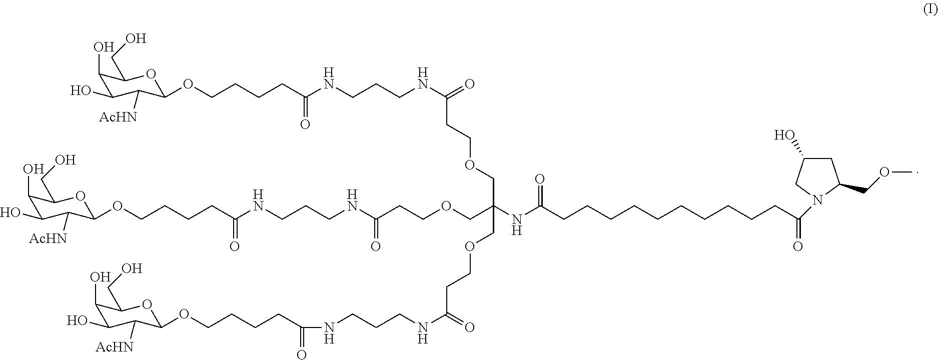 [SMILES: COC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

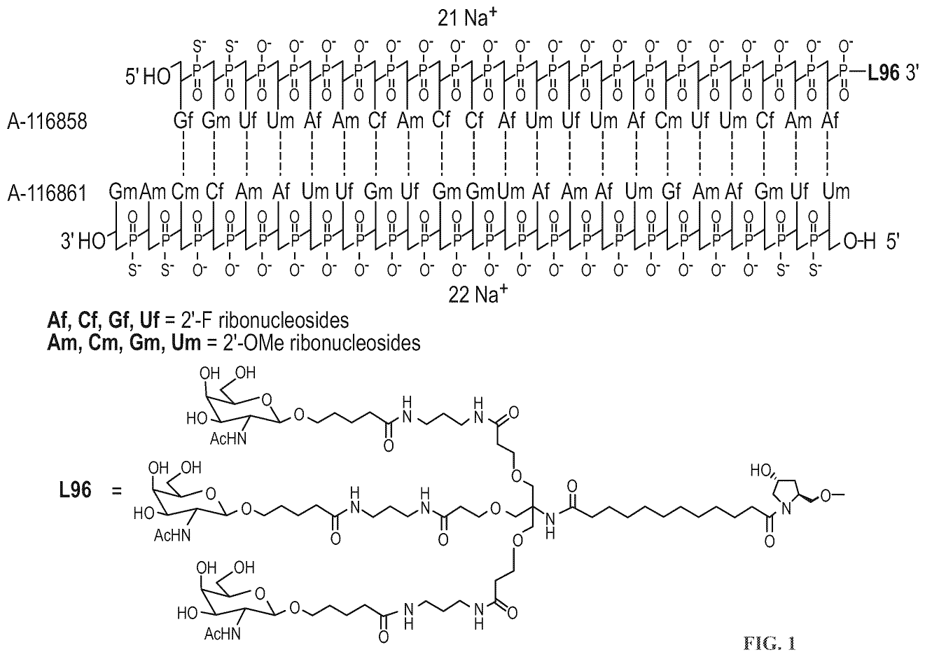

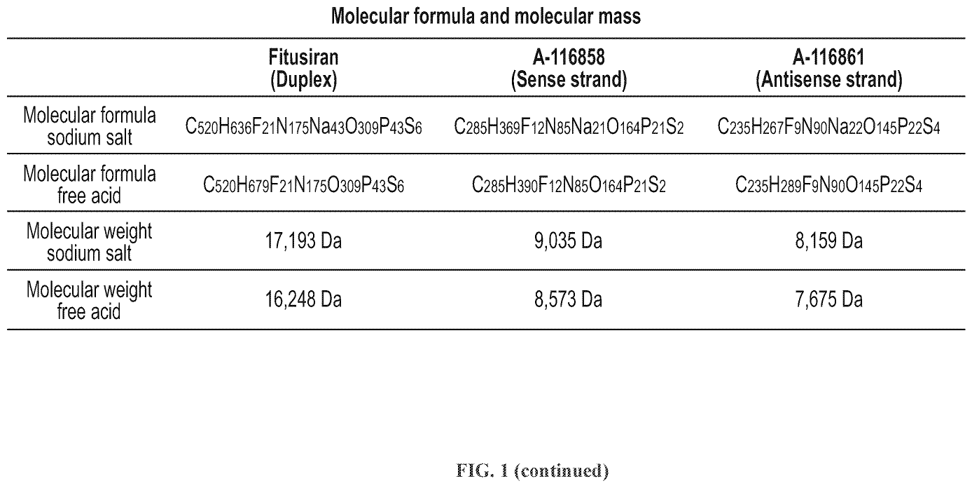

The expanded structural formula, molecular formula, and molecular weight of fitusiran are shown in FIG. 1. As used herein, the term 2′-fluoroadenosine is used interchangeably with the term 2′-deoxy-2′-fluoroadenosine; the term 2′-fluorocytidine is used interchangeably with the term 2′-deoxy-2′-fluorocytidine; the term 2′-fluoroguanosine is used interchangeably with the term 2′-deoxy-2′-fluoroguanosine, and the term 2′-fluorouridine is used interchangeably with the term 2′-deoxy-2′-fluorouridine.

The structure of fitusiran can also be described using the following diagram, wherein the X is O:

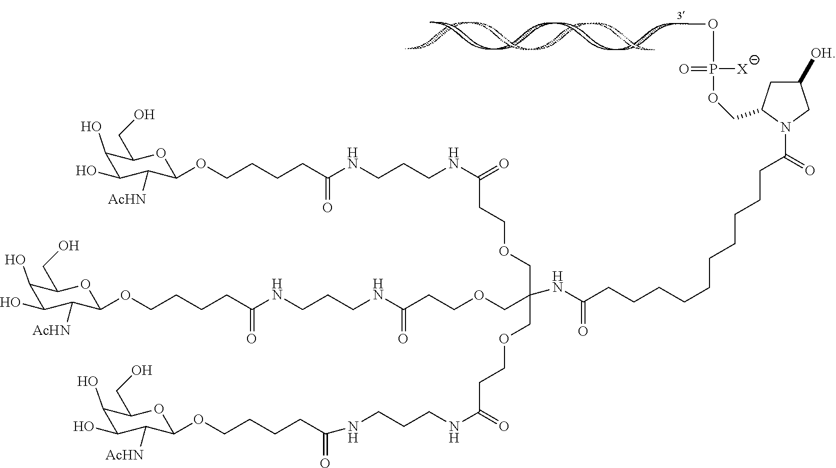

For use in the present treatment methods, fitusiran may be provided in a pharmaceutical composition comprising it and a pharmaceutically acceptable excipient. In certain embodiments, the dsRNA compound is in sodium salt form.

In some embodiments, fitusiran is provided in an aqueous solution at a concentration of 1 to 200 mg/mL (e.g., 50 to 150 mg/mL, 80 to 110 mg/mL, or 90 to 110 mg/mL, or 5 to 25 mg/mL, 7.5 to 20 mg/mL, or 10 to 15 mg/mL). As used herein, values intermediate to recited ranges and values are also intended to be part of this disclosure. In addition, ranges of values using a combination of any of recited values as upper and/or lower limits are intended to be included. In further embodiments, the pharmaceutical composition comprises fitusiran at a concentration of 5, 10, 12.5, 50, 75, 100, 125, 150, or 200 mg/mL. In certain embodiments, fitusiran is provided at a concentration of 100 mg/mL in an aqueous solution. In certain embodiments, fitusiran is provided at a concentration of 12.5 mg/mL in an aqueous solution. In certain embodiments, fitusiran is provided at a concentration of 6.25 mg/mL in an aqueous solution.

Unless otherwise indicated, a fitusiran weight recited in the present disclosure is the weight of fitusiran free acid (the active moiety), even though fitusiran is injected to patients subcutaneously in its sodium form (in an aqueous solution). For example, 100 mg/mL fitusiran means 100 mg of fitusiran free acid (equivalent to 106 mg fitusiran sodium, the drug substance) per mL.

In some embodiments, the pharmaceutical compositions comprise fitusiran in a phophate-buffered saline. The phosphate concentration in the solution may be 1 to 10 mM (e.g., 2, 3, 4, 5, 6, 7, 8, or 9 mM), with a pH of 6.0-8.0. The pharmaceutical compositions herein may include a preservative such as EDTA. Alternatively, the pharmaceutical compositions are preservative-free.

In particular embodiments, the fitusiran pharmaceutical composition is preservative-free and comprises, consists of, or consists essentially of 100 mg of fitusiran per mL of a 5 mM phosphate buffered saline (PBS) solution. The PBS solution is composed of sodium chloride, dibasic sodium phosphate (heptahydrate), and monobasic sodium phosphate (monohydrate). Sodium hydroxide solution and diluted phosphoric acid may be used to adjust the pH of the composition to about 7.0.

In particular embodiments, the fitusiran pharmaceutical composition is preservative-free and comprises, consists of, or consists essentially of 12.5 mg of fitusiran per mL of a 5 mM phosphate buffered saline (PBS) solution. The PBS solution is composed of sodium chloride, dibasic sodium phosphate (heptahydrate), and monobasic sodium phosphate (monohydrate). Sodium hydroxide solution and diluted phosphoric acid may be used to adjust the pH of the composition to about 7.0 (e.g., 7.1).

The pharmaceutical composition may be provided in a container (e.g., a vial or a syringe). The container may contain single or multiple doses. In some embodiments, the solution is administered to patients through subcutaneous injection. The solution can be stored at 2 to 30° C. (e.g., 2 to 8° C.). In some embodiments, the pharmaceutical composition is provided in a pre-filled, single-dose syringe. In some embodiments, the pre-filled, single-dose syringe comprises 20-50 mg (e.g., about 20 mg or about 30 mg) of fitusiran. In some embodiments, the vials are type I glass single-use vials; in further embodiments, the vials comprise at least 0.2 mL of the aqueous solution comprising fitusiran.

In some embodiments, fitusiran is provided in 30 mg vials (e.g., at 100 mg/mL), which may also be pooled to achieve a higher dose amount. These vials can also be used in half (half a vial) to achieve a dose amount of 15 mg.

In some embodiments, fitusiran is provided in 20 mg vials (e.g., at 100 mg/mL), which may also be pooed to achieve a high dose amount. These vials can also be used in half (half a vial) to achieve a dose amount of 10 mg.

In some embodiments, fitusiran is provided in 2.5 mg vials (e.g., at 12.5 mg/mL), which may be pooled to achieve a dose amount of, e.g., 5, 7.5, 10, 20, 30, or 50 mg. These vials can also be used in half (half a vial) to achieve a dose amount of 1.25 mg.

In some embodiments, fitusiran is provided in 1.25 mg vials (e.g., at 12.5 mg/mL), which may be pooled to achieve a dose amount of, e.g., 2.5, 5, 7.5, or 10 mg. In some embodiments, fitusiran is provided in 1.25 mg vials (e.g., at 6.25 mg/mL), which may be pooled to achieve a dose amount of, e.g., 2.5, 5, 7.5, or 10 mg.

In one embodiment, 80 mg of fitusiran is delivered in 0.8 mL (100 mg fitusiran/mL). In one embodiment, 50 mg of fitusiran is delivered in 0.5 mL (100 mg fitusiran/mL). In one embodiment, 20 mg of fitusiran is delivered in 0.5 mL (40 mg fitusiran/mL). In one embodiment, 30 mg of fitusiran is delivered in 0.5 mL (60 mg fitusiran/mL). In one embodiment, 10 mg of fitusiran is delivered in 0.5 mL (20 mg fitusiran/mL). In one embodiment, 7.5 mg of fitusiran is delivered in 0.5 mL (15 mg fitusiran/mL). In one embodiment, 5 mg of fitusiran is delivered in 0.5 mL (10 mg fitusiran/mL). In one embodiment, 2.5 mg of fitusiran is delivered in 0.5 mL (5 mg fitusiran/mL). In one embodiment, 1.25 mg of fitusiran is delivered in 0.5 mL (2.5 mg fitusiran/mL).

In particular embodiments, the fitusiran composition for subcutaneous injection contains fitusiran in a 5 mM phosphate buffered saline having 0.64 mM NaH₂PO₄, 4.36 mM Na₂HPO₄, and 84 mM NaCl at pH 7.0 or 7.1. In certain embodiments, the composition of fitusiran solution for subcutaneous injection is shown in Table 1A below.

TABLE 1A
Fitusiran Formulation

| | Composition | | | |
|---|---|---|---|---|
| | | | Per unit | Per unit |
| | | | (2 mL | (1 mL |
| | Percentage | Per ml | vial) | syringe) |
| Components | (%) | (mg) | (mg) | (mg) |
| Fitusiran (active moiety) | 10 | 100 | 80 | 80 |
| [equivalent to fitusiran | | [106] | [84.8] | [84.8] |
| sodium] | | | | |
| Sodium chloride | 0.49 | 4.909 | 3.927 | 3.927 |
| Dibasic sodium phosphate | 0.12 | 1.169 | 0.935 | 0.935 |
| (heptahydrate) | | | | |
| Monobasic sodium phosphate | <0.01 | 0.0885 | 0.0708 | 0.0708 |
| (monohydrate) | | | | |
| Phosphoric acid, concentrated | — | q.s. | q.s. | q.s. |
| | | pH 7.0 | pH 7.0 | pH 7.0 |
| Sodium hydroxide | — | q.s. | q.s. | q.s. |
| | | pH 7.0 | pH 7.0 | pH 7.0 |
| Water for injection | q.s. 100 | q.s. | q.s. | q.s. |
| | | 1 mL | 0.8 mL | 0.8 mL |

q.s.: quantum satis.

In certain embodiments, the composition of fitusiran solution for subcutaneous injection is shown in Table 1B below.

TABLE 1B
Fitusiran Formulations

| Components | Formulation 1 (mg) | Formulation 2 (mg) |
|---|---|---|
| Fitusiran | 12.5 | 100 |
| [Na salt] | [13.25] | [106] |
| NaH₂PO₄*H₂O | 0.388 | 0.0885 |
| Na₂HPO₄*7H₂O | 0.586 | 1.169 |
| NaCl | 8.7 | 4.909 |
| 0.1N NaOH | q.s. | q.s. |
| 0.1M H₃PO₄ | q.s. | q.s. |
| Purified water | Ad 1 mL | Ad 1 mL |

While the fitusiran dosage weight described herein refers to the weight of fitusiran free acid (active moiety), administration of fitusiran to patients herein refers to administration of fitusiran sodium (drug substance) provided in a pharmaceutically suitable aqueous solution (e.g., a phosphate buffered saline at a physiological pH).

### II. Therapeutic Use of Fitusiran

Fitusiran can suppress liver production of antithrombin (AT). In its role as an anti-coagulant, AT regulates hemostasis by directly targeting thrombin production or by inactivating uncomplexed FXa, which in turn reduces thrombin production (Quinsey et al., Int J Biochem Cell Biol. (2004) 36(3): 386-9). Fitusiran may be used to treat those who have impaired hemostasis. For example, fitusiran can be used as routine prophylaxis to treat hemophilia A or B in, or to prevent or reduce the frequency of bleeding episodes in, pediatric patients with or without inhibitors. In particular embodiments, fitusiran is used to treat pediatric patients (i.e., patients 1 to <12 year(s) of age), with hemophilia A or B (congenital factor VIII or factor IX deficiency) with or without inhibitors.

The present methods include administering to the hemophilia patient (e.g., a hemophilia A or B patient) in need thereof a therapeutically effective amount of fitusiran. “Therapeutically effective amount” refers to the amount of fitusiran that helps the patient to achieve a desired clinical endpoint. A desired clinical endpoint may be, for example, reduction of annual bleeding rates (ABR) to no more than 3, no more than 2, no more than 1, or zero. A desired clinical endpoint may also be, for example, reduction of annual spontaneous bleeding rate (AsBR) to no more than 1, and preferable zero.

The present treatment methods are based in part on the discovery that the risk of vascular thrombotic events in patients exposed to fitusiran may increase with lower AT levels. AT measurements can be performed by well-established methods, including both kinetic and chromogenic assays. One commonly used method is the INNOVANCE™ Antithrombin assay (Siemens Healthineers, Malvern, PA; U.S. FDA 510(k) #K081769). INNOVANCE™ is a chromogenic assay that quantifies functionally active AT in human citrated plasma based on the inhibition of an excess of factor Xa by AT. The assay may be performed by using an automated coagulation instrument (e.g., Siemens BCS® XP, Sysmex® CA-600 and CS Systems, or Atellica® COAG 360 System), and may be calibrated using Siemens standard human plasma (SHP) with a defined value of AT activity calibrated against World Health Organization (WHO) reference plasma. An equivalent assay is the Dade Behring Berichrom™ Antithrombin III assay (Dade Behring Marburg GmbH, Marburg, Germany; U.S. FDA 510(k) #K933125). Each measurement may be controlled by two independent controls (low and normal value) also calibrated against the WHO standard. The AT activity (%) in a plasma sample is calculated against the WHO reference plasma. 100% AT level is defined as 1 unit of antithrombin activity in 1 mL of reference plasma sample. The limit of detection of the INNOVANCE™ assay is 6.0% based on the assay's U.S. FDA 510(k) decision summary. AT levels range from about 80% to about 120% in the general population.

It has been observed that the risk of arterial thrombotic events among patients receiving fitusiran may increase with AT levels <10%. Thus, the patient's AT levels may be monitored and if necessary, the dose amounts of fitusiran may be adjusted.

In some embodiments, the patient has a steady state AT level within the desired range (e.g., 15-35%) but still has suboptimal bleeding control (e.g., having two or more treated bleeds within a 12-week period starting with the third fitusiran injection at the current dose, fitusiran will be dosed at the next higher level or at the next higher frequency (e.g., going from every two months or every eight weeks to every month or every four weeks). This dosing adjustment may be adjusted until the patient has no more than two treated bleeds within a 12-week period starting with the third fitusiran injection at the current dose while maintaining a steady state AT range of 15-35%.

In some embodiments, fitusiran is subcutaneously administered to the patient at a range of 1 mg to 50 mg. In further embodiments, fitusiran is subcutaneously administered to the patient at a range of 1 mg to 30 mg. In further embodiments, fitusiran is subcutaneously administered to the patient at a range of 1 mg to 20 mg. In further embodiments, fitusiran is subcutaneously administered to the patient at a range of 1 mg to 10 mg. In further embodiments, fitusiran is subcutaneously administered to the patient at a range of 1 mg to 5 mg. In certain embodiments, fitusiran is subcutaneously administered to the patient at 1 mg, 1.25 mg, 1.5 mg, 1.75 mg, 2 mg, 2.25 mg, 2.5 mg, 2.75 mg, 3 mg, 3.25 mg, 3.5 mg, 3.75 mg, 4 mg, 4.25 mg, 4.5 mg, 4.75 mg, 5 mg, 5.5 mg, 6 mg, 6.5 mg, 7 mg, 7.5 mg, 8 mg, 8.5 mg, 9 mg, 9.5 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, or 50 mg. In certain embodiments, fitusiran is subcutaneously administered every other month or every eight weeks, or every month or every four weeks, at, e.g., one of the aforementioned weight dosages.

In some embodiments, fitusiran is subcutaneously administered every other month or every eight weeks at 1.25 mg.

In some embodiments, fitusiran is subcutaneously administered every month or every four weeks at 1.25 mg.

In some embodiments, fitusiran is subcutaneously administered every other month or every eight weeks at 2.5 mg.

In some embodiments, fitusiran is subcutaneously administered every month or every four weeks at 2.5 mg.

In some embodiments, fitusiran is subcutaneously administered every other month or every eight weeks at 5 mg.

In some embodiments, fitusiran is subcutaneously administered every month or every four weeks at 5 mg.

In some embodiments, fitusiran is subcutaneously administered every other month or every eight weeks at 7.5 mg.

In some embodiments, fitusiran is subcutaneously administered every month or

every four weeks at 7.5 mg.

In some embodiments, fitusiran is subcutaneously administered every other month or every eight weeks at 10 mg.

In some embodiments, fitusiran is subcutaneously administered every month or every four weeks at 10 mg.

In some embodiments, fitusiran is subcutaneously administered every other month or every eight weeks at 20 mg.

In some embodiments, fitusiran is subcutaneously administered every month or every four weeks at 20 mg.

In some embodiments, fitusiran is subcutaneously administered every other month or every eight weeks at 30 mg.

In some embodiments, fitusiran is subcutaneously administered every month or

every four weeks at 30 mg.

In some embodiments, fitusiran is subcutaneously administered every other month or every eight weeks at 50 mg.

In some embodiments, fitusiran is subcutaneously administered every month or every four weeks at 50 mg.

Exemplary treatment protocols are further described below.

Pediatric Patients with a Higher Body Weight

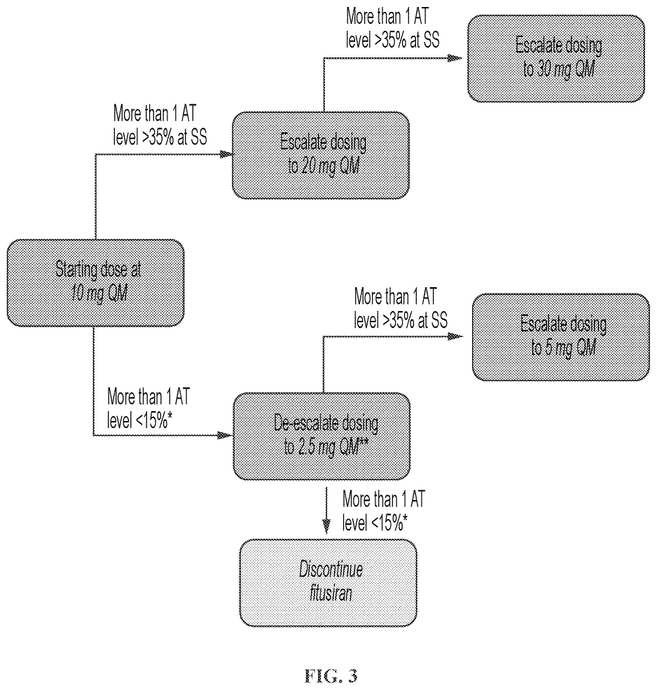

Patients with a body weight of 22 kg to <45 kg may begin with a starting dose of 10 mg fitusiran every month (or every four weeks). The patient's AT level may be monitored periodically (e.g., every one, two, three, four, five, six, seven, or eight weeks, or every one, two, three, four, five, or six months). An exemplary escalation and de-escalation scheme for patients in Cohort 1 is illustrated in FIG. 3.

In some embodiments, patients treated with a starting dose of 10 mg fitusiran every month (or every four weeks) de-escalate their fitusiran dose regimen. In some embodiments, upon the first AT level <15%, the patient has another AT activity level sample drawn within a month (e.g., within one or two weeks). If this result is <15%, this is considered the second AT<15%. Patients receiving fitusiran at a dose of 10 mg QM with more than 1 (e.g., 2) AT activity levels <15% will de-escalate to a dose of 2.5 mg fitusiran every month (or every four weeks). The patient may initiate treatment with the lower dose of fitusiran after their AT levels have returned to above 15%, e.g., ≥22%.

After de-escalating to a dose of 2.5 mg fitusiran every month, the patient's AT level may be again monitored periodically (e.g., every one, two, three, four, five, six, seven, or eight weeks, or every one, two, three, four, five, or six months). In some embodiments, upon the first AT level <15%, the patient has another AT activity level sample drawn within a month (e.g., within one or two weeks). If this result is <15%, this is considered the second AT<15%. Patients receiving fitusiran at a dose of 2.5 mg QM with more than 1 (e.g., 2) AT activity levels <15% may discontinue or pause fitusiran treatment.

Alternatively, after de-escalating to a dose of 2.5 mg fitusiran every month, the patient's AT level is again monitored periodically (e.g., every one, two, three, four, five, six, seven, or eight weeks, or every one, two, three, four, five, or six months). In some embodiments, upon the first steady state AT level >35%, the patient has another AT activity level sample drawn within a month (e.g., within one or two weeks). If this result is >35%, this is considered the second steady state AT >35%. Patients receiving fitusiran at a dose of 2.5 mg QM with more than 1 (e.g., 2) steady state AT activity levels >35% may escalate to a dose of 5 mg fitusiran once a month (or every four weeks).

In other embodiments, patients treated with a starting dose of 10 mg fitusiran every month (or every four weeks) may escalate their dose of fitusiran. The patient's AT level may be monitored periodically (e.g., every one, two, three, four, five, six, seven, or eight weeks, or every one, two, three, four, five, or six months). In some embodiments, upon the first steady state AT level >35%, the patient has another steady state AT activity level sample drawn within a month (e.g., within one or two weeks). If this result is >35%, this is considered the second steady state AT >35%. Patients receiving fitusiran at a dose of 10 mg QM with more than 1 (e.g., 2) steady state AT activity levels >35% may escalate to a dose of 20 mg fitusiran every month (or every four weeks).

After escalating to a dose of 20 mg fitusiran every month, the patient's AT level may be again monitored periodically (e.g., every one, two, three, four, five, six, seven, or eight weeks, or every one, two, three, four, five, or six months). In some embodiments, upon the first steady state AT level >35%, the patient has another steady state AT activity level sample drawn within a month (e.g., within one or two weeks). If this result is >35%, this is considered the second steady state AT >35%. Patients receiving fitusiran at a dose of 20 mg QM with more than 1 (e.g., 2) steady state AT activity levels >35% may escalate to a dose of 30 mg fitusiran once a month (or every four weeks).

In some embodiments, escalation is performed after AT activity levels at steady state remain >35% (see, e.g., FIG. 3).

Pediatric Patients with a Lower Body Weight

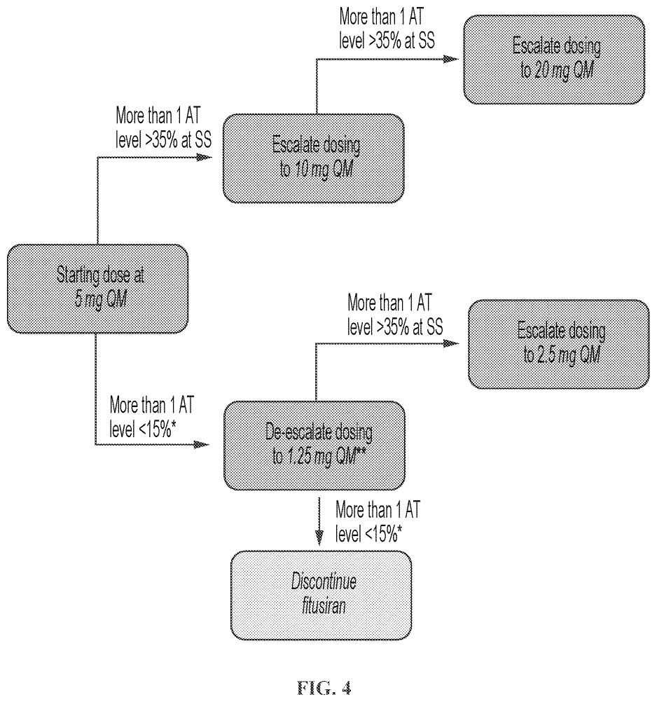

Patients with a body weight of 8 kg to <22 kg (Cohort 2) may begin with a starting dose of 5 mg fitusiran every month (or every four weeks). The escalation and de-escalation scheme for patients in Cohort 2 is illustrated in FIG. 4. The patient's AT level may be monitored periodically (e.g., every one, two, three, four, five, six, seven, or eight weeks, or every one, two, three, four, five, or six months).

In some embodiments, patients treated with a starting dose of 5 mg fitusiran every month (or every four weeks) may de-escalate their dose of fitusiran. In some embodiments, upon the first AT level <15%, the patient has another AT activity level sample drawn within a month (e.g., within one or two weeks). If this result is <15%, this is considered the second AT<15%. Patients receiving fitusiran at a dose of 5 mg QM with more than 1 (e.g., 2) AT activity levels <15% may de-escalate to a dose of 1.25 mg fitusiran every month (or every four weeks). The patient may initiate with the lower dose of fitusiran after their AT levels have returned to above 15%, e.g., ≥22%.

After de-escalating to a dose of 1.25 mg fitusiran every month, the patient's AT level may be again monitored periodically (e.g., every one, two, three, four, five, six, seven, or eight weeks, or every one, two, three, four, five, or six months). In some embodiments, upon the first AT level <15%, the patient has another AT activity level sample drawn within a month (e.g., within one or two weeks). If this result is <15%, this is considered the second AT<15%. Patients receiving fitusiran at a dose of 1.25 mg QM with more than 1 (e.g., 2) AT activity levels <15% may discontinue or pause fitusiran treatment.

Alternatively, after de-escalating to a dose of 1.25 mg fitusiran every month, the patient's AT level may again be monitored periodically (e.g., every one, two, three, four, five, six, seven, or eight weeks, or every one, two, three, four, five, or six months). In some embodiments, upon the first steady state AT level >35%, the patient has another steady state AT activity level sample drawn within a month (e.g., within one or two weeks). If this result is >35%, this is considered the second steady state AT >35%. Patients receiving fitusiran at a dose of 1.25 mg QM with more than 1 (e.g., 2) steady state AT activity levels >35% may escalate to a dose of 2.5 mg fitusiran once a month (or every four weeks).

In other embodiments, patients treated with a starting dose of 5 mg fitusiran every month (or every four weeks) may escalate their dose of fitusiran. The patient's AT level may be monitored periodically (e.g., every one, two, three, four, five, six, seven, or eight weeks, or every one, two, three, four, five, or six months). In some embodiments, upon the first steady state AT level >35%, the patient has another steady state AT activity level sample drawn within a month (e.g., within one or two weeks). If this result is >35%, this is considered the second steady state AT >35%. Patients receiving fitusiran at a dose of 5 mg QM with more than 1 (e.g., 2) steady state AT activity levels >35% may escalate to a dose of 10 mg fitusiran every month (or every four weeks).

After escalating to a dose of 10 mg fitusiran every month, the patient's AT level may again be monitored periodically (e.g., every one, two, three, four, five, six, seven, or eight weeks, or every one, two, three, four, five, or six months). In some embodiments, upon the first steady state AT level >35%, the patient has another steady state AT activity level sample drawn within a month (e.g., within one or two weeks). If this result is >35%, this is considered the second steady state AT >35%. Patients receiving fitusiran at a dose of 10 mg QM with more than 1 (e.g., 2) steady state AT activity levels >35% may escalate to a dose of 20 mg fitusiran once a month (or every four weeks).

In some embodiments, escalation is performed after AT activity levels at steady state remain >35% (see, e.g., FIG. 4).

In some embodiments, AT activity levels used for deciding whether to escalate a fitusiran dose amount or frequency are those measured at steady state (SS), i.e., once the patient's AT levels have been stabilized after fitusiran treatment. The SS is typically reached after two or three doses of fitusiran. AT measurements for dosing determination are taken at an appropriate interval (e.g., every four weeks or every eight weeks).

In the above dose finding regimens, the starting dose of 10 mg fitusiran QM or 5 mg fitusiran QM is discussed as an illustrative example. For example, a starting dose of fitusiran may be 20 mg QM, 10 mg Q2M, 7.5 mg Q2M, 5 mg Q2M, or 2.5 mg QM. Dose escalation and de-escalation can then be carried out accordingly from each starting dose. For example, a starting dose of 2.5 mg QM fitusiran can be escalated to 5 mg QM, 10 mg QM, 20 mg QM, 30 mg QM, or 50 mg QM, optionally sequentially in that order, or de-escalated to 2.5 mg Q2M or 1.25 QM.

An AT level of 10-35% (e.g., 10-25%, 15-35%, or 15-25%) is targeted to mitigate the risk of vascular thrombotic events while aiming to maintain a favorable benefit-risk balance for patients on fitusiran. Thus, so long as the patient reaches this targeted AT level, there is no need for the patient to receive a higher fitusiran dosage or more frequent dosing except as otherwise discussed herein (e.g., patients having bleeding events more often than a pre-set threshold). That is, he may remain on the current treatment regimen (i.e., maintenance regimen). For example, once the desired AT level is reached, the patient may be treated with a subcutaneous dose of fitusiran (e.g., 1.25-30 mg per dose) at an interval of, e.g., every one, two, three, four, five, six, seven, or eight weeks, or every one, two, three, or four months. In some embodiments, if the patient has two AT measurements of no greater than 35% while receiving 10 mg QM, he will maintain this dosing regimen, with no need to further escalate the dosage or dosing frequency. As another example, if the patient has two AT measurements of no greater than 35% while receiving 5 mg QM, he will remain on this dosing regimen, with no need to further escalate the dosage or dosing frequency (to, e.g., 10 mg QM or 20 mg QM). However, fitusiran treatment should be dose de-escalated (or discontinued if already de-escalated to the lowest permissible dose) if a patient has more than 1 (e.g., 2) AT measurements <15% (e.g., <10%) as a risk mitigation measure for vascular thrombotic events. In some embodiments, the exceptions may be that when the patients bleed more often than a pre-set threshold, they escalate the fitusiran dosage despite having a within-range (15-35%) AT activity level.

A pediatric patient with hemophilia A or B with or without inhibitors may be treated with a subcutaneous maintenance dose of fitusiran at 1-50 mg per dose every month (or every four weeks). In some embodiments of maintenance regimens, a patient with hemophilia A or B with or without inhibitors is treated with a subcutaneous dose of fitusiran at 50 mg per dose every month (or every four weeks). In some embodiments of maintenance regimens, a patient with hemophilia A or B with or without inhibitors is treated with a subcutaneous dose of fitusiran at 30 mg per dose every month (or every four weeks). In other embodiments, a patient with hemophilia A or B with or without inhibitors is treated with a subcutaneous dose of fitusiran at 20 mg every month (or every four weeks). In other embodiments, a patient with hemophilia A or B with or without inhibitors is treated with a subcutaneous dose of fitusiran 10 mg every month (or every four weeks). In some embodiments of maintenance regimens, a patient with hemophilia A or B with or without inhibitors is treated with a subcutaneous dose of fitusiran at 7.5 mg per dose every month (or every four weeks). In other embodiments, a patient with hemophilia A or B with or without inhibitors is treated with a subcutaneous dose of fitusiran at 5 mg every month (or every four weeks). In other embodiments, a patient with hemophilia A or B with or without inhibitors is treated with a subcutaneous dose of fitusiran at 2.5 mg every month (or every four weeks). In other embodiments, a patient with hemophilia A or B with or without inhibitors is treated with a subcutaneous dose of fitusiran at 1.25 mg every month (or every four weeks).

In some embodiments, patients may receive periodic (e.g., monthly or every four weeks) AT monitoring for 12 months following a change in fitusiran dosing regimen.

In some embodiments, once a patient stays on a maintenance regimen (e.g., 1.25 mg QM or Q4W, 2.5 mg QM or Q4W, 5 mg QM or Q4W, 7.5 mg QM or Q4W, 10 mg QM or Q4W, 20 mg QM or Q4W, 30 mg QM or Q4W, or 50 mg QM or Q4W), the patient may receive less frequent AT monitoring. For example, his AT level may be monitored every month, every two months, every three months, every four months, semi-annually, annually, or every two years.

### III. Patient Management

Patients on fitusiran are monitored for hemostasis parameters, e.g., coagulation parameters (D-dimer, prothrombin fragment 1+2, and fibrinogen) and for signs and symptoms of vascular thrombotic events. Such signs and symptoms may include, but are not limited to, severe or persistent headache, headache with nausea and vomiting, chest pain and/or tightness, coughing up blood, trouble breathing, abdominal pain, fainting or loss of consciousness, swelling or pain in the arms or legs, vision problems, weakness and/or sensory deficits, and changes in speech. An evaluation of signs and symptoms potentially consistent with vascular thrombosis should include appropriate imaging studies as applicable. For the diagnosis of cerebral venous sinus thrombosis magnetic resonance imaging venogram (MRV) or computed tomography venogram (CTV) is recommended.

If a patient develops thrombosis while on fitusiran, AT reversal may be administered in combination with a replacement factor or BPA and appropriate anticoagulation. AT reversal should follow labeled product recommendations for the prevention of perioperative thrombosis in patients with AT deficiency, and individualize patient doses to target 80-120% AT activity. The use of plasma derived AT may be preferable to recombinant AT, given its longer half-life.

Bleeding events in patients on fitusiran may be managed by on-demand administration of a replacement factor (recombinant or plasma-derived Factor VIII or Factor IX) or a BPA (e.g., fresh-frozen plasma (FFP); rFVIIa; and aPCC). The amount of the factor or BPA must be reduced in patients on fitusiran to prevent vascular thrombosis. See, e.g., WO 2019/014187. Management of bleeding episodes in pediatric patients who are on prophylactic fitusiran treatment is described in further detail in Example 1. See, e.g., Table 4, infra.

Additional definitions of terminology and exemplary embodiments are described in the Examples and are incorporated by reference herein.

Unless otherwise defined herein, scientific, and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of hematology, medicine, medicinal and pharmaceutical chemistry, and cell biology described herein are those well-known and commonly used in the art. Further, unless otherwise required by context, singular terms shall include pluralities and plural terms shall include the singular. Throughout this specification and aspects, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain aspects, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

According to the present disclosure, back-references in the dependent claims are meant as short-hand writing for a direct and unambiguous disclosure of each and every combination of claims that is indicated by the back-reference. Further, headers herein are created for ease of organization and are not intended to limit the scope of the claimed invention in any manner.

In order that this invention may be better understood, the following examples are set forth. These examples are for purposes of illustration only and are not to be construed as limiting the scope of the invention in any manner.

### EXAMPLES

### Example 1: Clinical Trial Protocol for an Open-Label Study of Fitusiran Prophylaxis in Male Pediatric Subjects Aged 1 to Less than 12 Years with Hemophilia A or B

This Example describes the protocol for an open label, multinational study of fitusiran prophylaxis in male pediatric participants aged 1 to <12 years with severe hemophilia A or hemophilia B with inhibitory antibodies to FVIII or FIX. People diagnosed with severe hemophilia A or hemophilia B with inhibitory antibodies are eligible for enrollment as long as they meet all inclusion and none of the exclusion criteria.

The objective of the study is to confirm appropriate starting, escalation, and de-escalation doses of fitusiran when administered to male pediatric participants (aged 1 to <12 years on Day 1). The study consists of a screening period of up to 60 days (may be extended in exceptional circumstances, after consultation with the Sponsor) followed by a fitusiran treatment period during which participants will receive fitusiran starting dose, administered subcutaneously every 4 weeks.

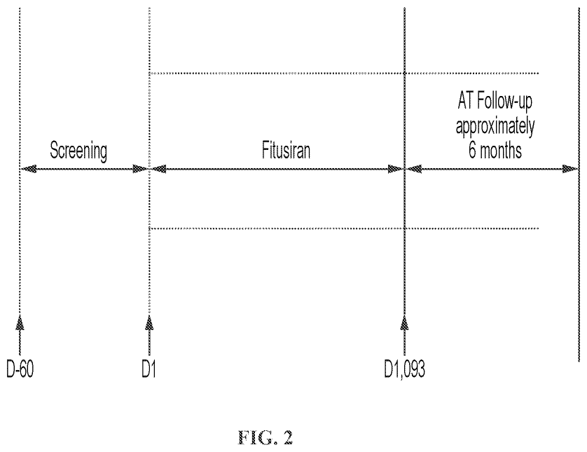

Participants who experience more than one antithrombin activity level less than 15% will be allowed to de-escalate to a lower fitusiran dose. Participants with more than 1 antithrombin activity level less than 15% on that lower dose must permanently discontinue fitusiran. If a participant's steady state antithrombin activity levels on the starting dose remain above 35%, the participant will be allowed to dose escalate to higher fitusiran doses. The study schema is depicted in FIG. 2. The doses administered will be based on 2 weight-based groups, Cohort 1 and Cohort 2, as further described below.

Throughout the study, participants may receive on-demand treatment for breakthrough bleeding episodes with BPAs as per the bleeding management guidelines as further described below.

Approximately 32 pediatric participants with severe hemophilia A or B, with inhibitory antibodies to FVIII or FIX, and receiving either on-demand or prophylaxis treatment with BPA, will enroll in the study (approximately 12 participants in Cohort 1 and 20 participants in Cohort 2). In Cohort 2, about half of the participants should weigh between 8 kg to <16 kg.

Once all the participants in the 22 kg to <45 kg weight cohort (Cohort 1) have received 3 consecutive injections of fitusiran starting dose administered every 4 weeks, the independent data monitoring committee (DMC) will review available safety and PD data from this first cohort, up until at least 4 weeks after the last dose. Endorsement from the DMC to proceed will trigger enrollment of participants who weigh 8 kg to <22 kg. Once all participants in the 8 kg to <22 kg weight cohort (Cohort 2) have received 3 consecutive injections of fitusiran administered every 4 weeks, the DMC will review available safety and PD data from both cohorts, up until at least 4 weeks after the last dose in the second cohort. The DMC will also review available safety and PD data for the fitusiran escalation and de-escalation doses, as well as the available PK data for the starting dose, once all participants in the concerned cohort have received injections of their respective dose.

Participants who complete the 12 weeks of treatment on starting, escalation or de-escalation doses, may be eligible to continue treatment with fitusiran administered every 4 weeks as part of this study or as participant in an open-label extension (OLE) study when available. Participants who remain on fitusiran will continue in the study until the completion of all dosing cohorts and until the pediatric dosing has been finalized. Home injection of fitusiran may be possible beginning at Week 56 where permitted by national and local regulations if participants and/or caregiver(s) complete training and meet eligibility requirements.

For participants who are deriving clinical benefit and remain on treatment, their estimated time in the study is up to 160 weeks. Duration in the study could be shorter if an OLE study is available prior to 160 weeks, and the participant is eligible, and the caregiver chooses to have the participant roll over into an OLE study.

Participants who stop fitusiran treatment will need to be monitored until their AT activity level return to approximately 60% per the central laboratory, or per Investigator discretion in consultation with the study Medical Manager. This might take up to 24 weeks.

During the study, pharmacodynamic (PD) and pharmacokinetic (PK) data as well as safety/tolerability data will be collected. Blood sampling times for PD is shown in Table 2.

TABLE 2
Schedule of Activities

| | Study Visit (Week) | | | | | | | | | | | | |
|---|---|---|---|---|---|---|---|---|---|---|---|---|---|
| | | Fitusiran Treatment Period^{a} | | | | | | | | | | | |
| | Screen- | Base- | Week | Week | Week | Week | Week | Week | Week | Week | Week | Week | Week |
| | ing | line | 2 | 4 | 8 | 12 | 16 | 20 | 24 | 28 | 32 | 36 | 40 |
| | Study Day (±Visit | | | | | | | | | | | | |
| | Window [Days]) | | | | | | | | | | | | |
| | Day | | Day | Day | Day | Day | Day | Day | Day | Day | Day | Day | Day |
| | −60 | Day | 15 ± | 29 ± | 57 ± | 85 ± | 113 ± | 141 ± | 169 ± | 197 ± | 225 ± | 253 ± | 281 ± |
| | to −1 | 1 | 3 | 7 | 7 | 7 | 7 | 7 | 7 | 7 | 7 | 7 | 7 |
| Enrollment | | X | | | | | | | | | | | |
| Fitusiran | | X | | X | X | X | X | X | X | X | X | X | X |
| adminis- | | | | | | | | | | | | | |
| tration^{f} | | | | | | | | | | | | | |
| Physical | X | X | X | X | X | X | X | X | X | | | X | |
| exami- | | | | | | | | | | | | | |
| nation^{g} | | | | | | | | | | | | | |
| Body | X | X | X | X | X | X | X | X | X | | | X | |
| weight | | | | | | | | | | | | | |
| and | | | | | | | | | | | | | |
| height^{h} | | | | | | | | | | | | | |
| Vital | X | X | X | X | X | X | X | X | X | | | X | |
| signs | | | | | | | | | | | | | |
| FibroScan | X | | | | | | | | | | | | |
| OR | | | | | | | | | | | | | |
| FibroTest | | | | | | | | | | | | | |
| and | | | | | | | | | | | | | |
| APRIⁱ | | | | | | | | | | | | | |
| Plasma | | X | | | X | | X | | X | | | | |
| TG | | | | | | | | | | | | | |
| Coagu- | X | X | X | X | X | X | X | X | X | | | X | |
| lation | | | | | | | | | | | | | |
| Nijmegen- | X | | | | | | | | | | | | |
| Modified | | | | | | | | | | | | | |
| Bethesda | | | | | | | | | | | | | |
| assay | | | | | | | | | | | | | |
| (Inhibitor | | | | | | | | | | | | | |
| status) | | | | | | | | | | | | | |
| AT | X | X | X | X | X | X | X | X | X | X | X | X | X |
| activity | | | | | | | | | | | | | |
| level^{j, k, l} | | | | | | | | | | | | | |
| FVIII/ | X | | | | | | | | | | | | |
| FIX | | | | | | | | | | | | | |
| levels^{k} | | | | | | | | | | | | | |
| Thrombo- | X | | | | | | | | | | | | |
| philia | | | | | | | | | | | | | |
| screening | | | | | | | | | | | | | |
| Serum | X | X | | X | X | X | X | X | X | | | X | |
| chemistry | | | | | | | | | | | | | |
| Liver | X | X | X | X | X | X | X | X | X | X | X | X | X |
| function | | | | | | | | | | | | | |
| tests^{m} | | | | | | | | | | | | | |
| Antidrug | | X | | | | X | | | X | | | X | |
| antibodies^{l} | | | | | | | | | | | | | |
| Hema- | X | X | | X | X | X | X | X | X | | | X | |
| tology | | | | | | | | | | | | | |
| Hepatic | X | | | | | | | | | | | | |
| tests^{m} | | | | | | | | | | | | | |
| Plasma | | X | | | | X | | | | | | | |
| PK | | | | | | | | | | | | | |

| | |
|---|---|
| Bleed | Review with participant/parent(s)/legal guardian |
| management | (hereinafter referred to as the “parent”) at each visit and |
| review | contact participant/parent every 2 weeks (±4 days) between visits |
| Bleeding | Review with participant/parent at each visit and contact |
| episodes/ | participant/parent every 2 weeks (±4 days) between visits |
| eDiary | |

| Adverse | Continuous | | | | | | | | | | | |
|---|---|---|---|---|---|---|---|---|---|---|---|---|
| events | | | | | | | | | | | | |
| Concomitant | Continuous | | | | | | | | | | | |
| medication | | | | | | | | | | | | |
| | Study Visit (Week) | | | | | | | | | | | |
| | Fitusiran Treatment Period^{a} | | | | | | | | | | EOS/ | |
| | | | | | | | | | | EOT | ET^{b} | |
| | Week | Week | Week | Week | Week | Week | Week | Week | Week | Week | Week | AT |
| | 44 | 48 | 52 | 60 | 72 | 84 | 96 | 108 | 132 | 156 | 160 | F/U^{b, c} |
| | Study Day (±Visit | | | | | | | | | | | |
| | Window [Days]) | | | | | | | | | | | |
| | Day | Day | Day | Day | Day | Day | Day | Day | Day | Day | Day | Monthly |
| | 309 ± | 337 ± | 365 ± | 421 ± | 505 ± | 589 ± | 673 ± | 757 ± | 925 ± | 1,093 ± | 1,121 ± | until AT |
| | 7 | 7 | 7 | 7 | 7 | 7 | 7 | 7 | 7 | 7 | 7 | ~60% |
| Enrollment | | | | | | | | | | | | |
| Fitusiran | X | X | X | Every 28 | | | | | | X | | |
| adminis- | | | | days ± 7 days | | | | | | | | |
| tration^{f} | | | | | | | | | | | | |
| Physical | | X | | X | X | X | X | X | X | X | X | |
| exami- | | | | | | | | | | | | |
| nation^{g} | | | | | | | | | | | | |
| Body | | X | | X | X | X | X | X | X | X | X | |
| weight | | | | | | | | | | | | |
| and | | | | | | | | | | | | |
| height^{h} | | | | | | | | | | | | |
| Vital | | X | | X | X | X | X | X | X | X | X | |
| signs | | | | | | | | | | | | |
| FibroScan | | | | | | | | | | | | |
| OR | | | | | | | | | | | | |
| FibroTest | | | | | | | | | | | | |
| and | | | | | | | | | | | | |
| APRIⁱ | | | | | | | | | | | | |
| Plasma | | | | | | | | | | | | |
| TG | | | | | | | | | | | | |
| Coagu- | | X | | X | X | X | X | X | X | X | X | |
| lation | | | | | | | | | | | | |
| Nijmegen- | | | | | | | | | | | X | |
| Modified | | | | | | | | | | | | |
| Bethesda | | | | | | | | | | | | |
| assay | | | | | | | | | | | | |
| (Inhibitor | | | | | | | | | | | | |
| status) | | | | | | | | | | | | |
| AT | X | X | X | X | X | X | X | X | X | X | X | X |
| activity | | | | | | | | | | | | |
| level^{j, k, l} | | | | | | | | | | | | |
| FVIII/ | | | | | | | | | | | | |
| FIX | | | | | | | | | | | | |
| levels^{k} | | | | | | | | | | | | |
| Thrombo- | | | | | | | | | | | | |
| philia | | | | | | | | | | | | |
| screening | | | | | | | | | | | | |
| Serum | | X | | X | X | X | X | X | X | X | X | |
| chemistry | | | | | | | | | | | | |
| Liver | X | X | X | X | X | X | X | X | X | X | X | |
| function | | | | | | | | | | | | |
| tests^{m} | | | | | | | | | | | | |
| Antidrug | | X | | X | | X | | X | | X | X | |
| antibodies^{l} | | | | | | | | | | | | |
| Hema- | | X | | X | X | X | X | X | X | X | X | |
| tology | | | | | | | | | | | | |
| Hepatic | | | | | | | | | | | | |
| tests^{m} | | | | | | | | | | | | |
| Plasma | | | | | | | | | | | | |
| PK | | | | | | | | | | | | |

| | |
|---|---|
| Bleed | Review with participant/parent(s)/legal guardian |
| management | (hereinafter referred to as the “parent”) at each visit and |
| review | contact participant/parent every 2 weeks (±4 days) between visits |
| Bleeding | Review with participant/parent at each visit and contact |
| episodes/ | participant/parent every 2 weeks (±4 days) between visits |
| eDiary | |
| Adverse | Continuous |
| events | |
| Concomitant | Continuous |
| medication | |

Abbreviations: ADA = antidrug antibody; AE = adverse event; APRI = AST to platelet ratio index; AT = antithrombin; BPA = bypassing agent; eDiary = electronic diary; EOS = End of Study; EOT = End of Treatment; ET = Early Termination; FVIII = factor VIII; FIX = factor IX; F/U = follow-up; LFT = liver function test; PK = pharmacokinetic; SAE = serious adverse event, ULN = upper limit of normal; TG = thrombin generation.
Notes:
In exceptional circumstances, screening may be extended beyond the 60-day window after consultation with the Sponsor. In this case, some assessments performed at the beginning of screening may have to be repeated.
Rescreening of participants is permitted after consultation with the study Medical Manager.
Participants in Cohort 1 who were receiving fitusiran prior to the dose hold will receive a new participant number and will resume dosing starting with the baseline visit, at a fitusiran dose of 10 mg administered SC once every 4 weeks.
When scheduled at the same time points, vital signs will be performed before the physical examinations and blood sample collections.
Unless otherwise specified, assessments on dosing days are pre-dose.
Blood samples will be obtained as blood volume permits and will not exceed the blood volume collection limit of 2 mL/kg for any given 24-hour period and 7 mL/kg over an 8-week period. Sampling is prioritized for safety laboratory tests (LFT, hematology, biochemistry), AT, ADA, and then PK.
^{a}Fitusiran as prophylaxis treatment and permitted on-demand use of BPAs for treatment of breakthrough bleeding episodes. Participants may continue their routine BPA regimen (including BPA prophylaxis for participant under prophylaxis) for up to the first 7 days following the first injection of fitusiran starting dose. Beginning at Day 8, BPA treatment for bleeding episode management should follow bleeding management guidelines.
^{b}Participants who discontinue fitusiran dosing for any reason will need to complete the EOS/ET visit and the AT F/U visits. They may receive treatment consistent with bleed management guidelines until their AT activity level returns to approximately 60%.
^{c}Participants not continuing fitusiran treatment in this study or not enrolling in an OLE study will complete assessments through the AT F/U visits at monthly intervals following final fitusiran dose until AT activity levels return to approximately 60%, per the central laboratory, or per Investigator discretion in consultation with the study Medical Manager.
^{d}Complete medical history/disease history (i.e., including bleeding episode and treatment history in the previous 6 months) to be recorded at screening.
^{e}eDiary training will be completed at the clinic at screening. Refresher training should also be provided at the Baseline visit
^{f}SC administration of fitusiran as per the Pharmacy Manual and will continue every 28 days ± 7 days when study site visits and assessments occur quarterly and every 6 months. Training on home injection will occur at the study site. Home injection may occur as per conditions and instructions, but not earlier than Week 56.
^{g}A complete physical examination will be performed at screening only; a directed physical examination will be performed at all other visits.
^{h}Height and weight will be recorded at all visits.
ⁱHepatitis C virus antibody-positive participants only. FibroScan where available, otherwise FibroTest and APRI.
^{j}After Week 52 visit, monthly AT testing will need to be resumed for at least 12 months every time there is a change in the fitusiran dose. At each dose level, upon the first AT level <15%, the participant must have another AT activity level sample drawn within 1 week of site receipt of the results. If this result is <15%, this will be considered the second AT activity level <15%.
^{k}After specified analyses are run, residual samples (excluding ADA and PK samples) may be stored for up to 25 years from the completion of the clinical study report, and used for further study of biomarkers related to hemophilia and associated conditions, investigation of emerging safety issues, or the development of fitusiran. PK and ADA samples will be disposed off after CSR finalization unless data indicate that additional analyses may be required which could be conducted at a later time. Samples drawn from central lines may be excluded from TG analysis.
^{l}On fitusiran dosing days, pre-dose samples will be collected within 4 hours prior to dosing.
^{m}LFT results may be obtained up to 7 days before the study site visit on which fitusiran dosing is scheduled. LFTs performed within 7 days of Day 1 will only be used to inform dosing on Day 1 and do not need to be used to confirm eligibility. LFTs can be analyzed locally, but if a local assessment is performed, a serum chemistry sample must also be collected for analysis at the central laboratory. LFT results will be obtained prior to receiving monthly fitusiran dosing.
Monthly pre-dose LFT testing is not required after Week 52 for participants who have met the criteria for quarterly pre-dose LFT monitoring described as follows:
Did not have any alanine transaminase elevation >3 × ULN persisting for ≥2 months at any time during fitusiran treatment; and
Must not have had any fitusiran doses held due to LFT elevations during the first 12 months of this study.

Blood sampling times for PK are shown in Table 3.

TABLE 3
Pharmacokinetic time points for pediatric participants
Pharmacokinetic time points for all participants

| Study day | Protocol time (hh:mm) | Plasma sample |
|---|---|---|
| Day 1 | 04:00 (±30 minutes)^{a} | X |
| Day 85 (±7 days) | Pre-dose | X |
| | (within 240 minutes before dosing)^{b} | |

^{a}The 4-hour timepoint corresponds to the median time to maximum concentration in adults.
^{b}The pre-dose timepoint corresponds to the minimum concentration after the third dose.
Note:
Time 00:00 is time of dosing.

Throughout the study, participants may receive on demand treatment for breakthrough bleeding episodes with BPAs as per the bleeding management guidelines further described below. Investigators will establish and provide instructions for an individualized bleed management plan based on the guidelines in Table 4 for each participant.

TABLE 4
Bleed management dosing guidelines by specific product

| | | Factor IX | Factor IX | | |
|---|---|---|---|---|---|
| | Factor | standard | extended | | Recombinant |
| | VIII | half-life | half-life | aPCC | factor VIIa |
| Recommended | 10 IU/kg | 20 IU/kg | 20 IU/kg | 30 U/kg | ≤45 μg/kg |
| single dose | | | | | |
| Single dose | 20 IU/kg | 30 IU/kg | 30 IU/kg | 50 U/kg | 45 μg/kg |
| should not | | | | | |
| exceed | | | | | |
| Repeat dose | Mandatory to call the study site prior to second dose | | | | Mandatory to call |
| instructions | Consider evaluation and treatment at the study site | | | | study site prior to |
| | | | | | third dose |
| | Should not | Should not | Should not | Should not | Should not |
| | repeat in less | repeat in less | repeat in less | repeat in less | repeat in less |
| | than 24 hours | than 24 hours | than 5-7 days | than 24 hours | than 2 hours |

| | |
|---|---|
| Should be seen at study site within 48-72 hours if | Should be seen at |
| more than 2 doses are required | study site within |

48-72 hours if more
than 3 doses are
required

For situations requiring higher doses, more frequent administration,
multiple repeated doses, discussion with study Medical Manager and
Clinical Advisor is recommended, and AT replacement should be
considered. Do not use antifibrinolytics in combination with factor or
BPA.

Details of bleeding episodes and doses of BPAs administered during the conduct of the study will be recorded in an electronic diary (eDiary).

### Scientific Rationale

A subcutaneous therapy that can effectively and safely prevent or reduce the frequency of bleeding episodes in patients with hemophilia A or B, including those with inhibitors, may reduce treatment burden, improve clinical outcomes, and enhance quality of life, especially for the pediatric patient population. In this patient category, patients with inhibitors appear to have the highest unmet therapeutic needs, and accordingly, enrollment to this dose confirmation study will be limited to patients with hemophilia A or B, with inhibitors.

The current study intends to confirm the appropriate doses of fitusiran to use in pediatric participants aged 1 to <12 years who are within the two weight categories of 22 kg to <45 kg, and 8 kg to <22 kg. This will be based on the level of AT activity, and safety and tolerability data.

Modeling and simulations were used to identify the dose at which participants within a particular weight cohort would be able to maintain an AT activity level ≥15%. The model was created taking into consideration the preclinical data and was based on adult dosing data and simulated approximately 1000 and 2000 pediatric participants for weight category of 22 kg to <45 kg, and 8 kg to <22 kg, respectively. In order to optimize the safety of the participants in this confirmatory study, the participants who are the heaviest (22 kg to <45 kg) will be dosed first with the 10 mg starting dose. Only when the DMC has evaluated available data from the largest cohort and deemed it appropriate will the next weight cohort be dosed.

### Dose Justification

The selection of the pediatric doses for specific body weight ranges are based on population PK/PD modelling. To take into consideration the lower body weights of pediatric participants 1 to <12 years of age, an adult PK model was updated using nonhuman primate AT lowering response data, and the updated model was then used to predict doses for the pediatric study.

Following the change in the fitusiran dose and regimen in the adult/adolescent studies, which was introduced as a risk mitigation measure for vascular thrombotic events, an update of the fitusiran dosing was implemented in this study. As the risk of vascular thrombotic events is thought to be increased in the setting of low AT activity levels, starting doses of 10 mg (Cohort 1) or 5 mg (Cohort 2) administered SC once every 4 weeks were selected to minimize the occurrence of AT activity levels below 10%. On these starting doses, if a participant has more than 1 AT level <15% (within a 12-month period), the participant can be de-escalated to a lower dose. If the participant has more than 1 AT level <15% (within a 12-month period) on that lower dose, he will be required to permanently discontinue fitusiran. Based on modeling and simulations, less than 1% of participants are expected to discontinue from fitusiran treatment based on their AT activity levels with this lower dose. The starting doses of 10 mg and 5 mg are projected by modeling and simulations to result in antithrombin levels between >15 to ≤35% in more than half of the participants in both Cohorts 1 and 2. If a participant has 2 steady state AT levels above 35% of the starting dose, the participant will be dose escalated to 20 mg (Cohort 1) or 10 mg (Cohort 2) administered SC once every 4 weeks. The escalation doses of 20 mg and 10 mg are projected to result in antithrombin levels between >15 to ≤35% in the majority of participants who had AT values of >35% at the starting doses of 10 mg and 5 mg. Further escalation to 30 mg (Cohort 1) and 20 mg (Cohort 2) administered SC once every 4 weeks will be allowed in case such escalation is needed.

In addition to the above, dose escalation will be allowed based on clinical criteria, with a goal to achieve adequate efficacy while maintaining AT levels above 15%.

### Objectives and Endpoints

The primary objective of the study is to confirm appropriate dose levels of fitusiran when administered to male pediatric participants (ages 1 to <12 years of age) with severe hemophilia A or B. The primary endpoint is to characterize the AT activity at the optimal therapeutic dose.

The secondary objects are to characterize the safety and tolerability and to determine fitusiran plasma concentrations at selected timepoints. The secondary endpoints are incidence, severity, seriousness, and relatedness of adverse events (AEs); and plasma concentrations in samples collected at 4 h post-dose on Day 1 (corresponding to median time to maximum concentration in adults) and at pre-dose on Day 85 (corresponding to minimum concentration after the third dose).

The tertiary/exploratory objectives are to characterize the frequency of bleeding episodes while receiving fitusiran treatment and to characterize immunogenicity. The tertiary/exploratory endpoints are annualized Bleeding Rate (ABR) from Day 1 up to the AT analysis timepoint at the optimal therapeutic dose, from the AT analysis timepoint up to the Week 160 end of study (EOS) visit, and overall Treatment Period; and incidence and titer of antidrug antibodies.

### Enrollment

Enrollment will be staggered by two bodyweight-based cohorts. Participants who weigh 22 kg to <45 kg (Cohort 1) will enroll first to receive 3 doses of fitusiran 10 mg administered every 4 weeks and participants who weigh 8 kg to <22 kg (Cohort 2) will enroll second to receive 3 doses of fitusiran 5 mg administered every 4 weeks. Fitusiran dose will be escalated to 20 mg administered every 4 weeks (for Cohort 1) or 10 mg administered every 4 weeks (for Cohort 2) for participants who have 2 steady state AT values greater than 35%. Participants who have 2 steady state AT values greater than 35% on the first escalated dose can be further escalated to 30 mg every 4 weeks (for Cohort 1) and 20 mg every 4 weeks (for Cohort 2).

### Study Population

Participants are eligible to be included in the study only if all of the following criteria apply:

### Age

I.01. Participant must be 1 to <12 years of age at the time of enrollment.

### Type of Participant and Disease Characteristics

I.02 Severe hemophilia A or B (FVIII <1% or FIX ≤2%)
I.03 Participants must have inhibitory antibodies to FVIII or FIX and must meet one of the following Nijmegen-modified Bethesda assay results criteria:
   Inhibitor titer of ≥0.6 BU/mL at screening, OR
   Inhibitor titer of <0.6 BU/mL at screening with medical record evidence of 2 consecutive titers ≥0.6 BU/mL, OR
   Inhibitor titer of <0.6 BU/mL at screening with medical record evidence of 1 inhibitor titer ≥0.6 BU/mL and a history of anamnestic response or severe allergic reaction (anaphylaxis or nephrotic syndrome).
I.04 Adequate peripheral venous access, as determined by the Investigator, to allow the blood draws required by the study protocol.

### Weight

I.05 Weight requirements at the time of enrollment:
   a) Cohort 1: Weight of 22 to <45 kg
   b) Cohort 2: Weight of 8 to <22 kg

### Sex

I.06 Male

There are no contraceptive requirements for this study except where required by local regulations.

Participants are to be excluded from the study if any of the following criteria apply:

### Medical Conditions

E.01 Known co-existing bleeding disorders other than hemophilia A or B, i.e., von Willebrand disease, additional factor deficiencies, or platelet disorders.
E.02 AT activity <60% at screening, as determined by central laboratory analysis.
E.03 Presence of clinically significant liver disease, or as indicated by any of the conditions below:
   a) International normalized ratio (INR)>1.2;
   b) Alanine aminotransferase (ALT) and/or aspartate aminotransferase (AST)>2×ULN reference range;
   c) Total bilirubin >ULN (>2×ULN in participants with Gilbert's syndrome);
   d) History of portal hypertension, esophageal varices, or hepatic encephalopathy;
   e) Presence of ascites by physical examination.
E.04 Hepatitis C virus antibody positive, except participants with a history of HCV infection who meet both of the following conditions:
   a) Completed curative treatment at least 12 weeks prior to enrollment and attained sustained virologic response as documented by a negative HCV ribonucleic acid (RNA) at screening, or they have spontaneously cleared the infection as documented by negative HCV RNA at screening.
   b) No evidence of cirrhosis according to one of the following assessments:
      FibroScan <9 kPa (where available), or
      FibroTest score <0.36 and AST to platelet ratio index (APRI)<1 (if FibroScan unavailable) (Tokuhara et al., PLOS One. (2016) 11(11):e016668315; de Lédinghen et al., J Pediatr Gastroenterol Nutr. (2007) 45(4): 443-50)
E.05 Presence of acute hepatitis, ie, hepatitis A, hepatitis E.
E.06 Presence of acute or chronic hepatitis B infection (IgM antibody to hepatitis B core antigen [anti-HBc IgM] positive or hepatitis B surface antigen [HBsAg] positive)*.
   As confirmed by central laboratory assessment of HBsAg, anti-HBc IgM and Total hepatitis B core antibody (Total anti-HBC).
E.07 Platelet count≤100,000/μL.
E.08 Presence of acute infection at screening, not including minor viral syndromes at the discretion of the Investigator in consultation with the study Medical Manager.
E.09 Known to be human immunodeficiency virus (HIV) positive with cluster of differentiation (CD) 4 count <400 cells/μL.
E.10 Estimated glomerular filtration rate≤45 mL/min/1.73 m² (using the Schwartz formula).
E.11 Co-existing thrombophilic disorder, as determined by presence of any of the below as identified at central laboratory:
   a) Factor V Leiden mutation (homozygous or heterozygous),
   b) Protein S deficiency,
   c) Protein C deficiency, and
   d) Prothrombin mutation (G20210A; homozygous and heterozygous).
E.12 History of antiphospholipid antibody syndrome.
E.13 History of arterial or venous thromboembolism, unrelated to an indwelling venous access.
E.14 Any condition (e.g., medical concern), which in the opinion of the Investigator, would make the participant unsuitable for dosing on Day 1 or which could interfere with the study compliance, the participant's safety and/or the participant's participation in the completion of the treatment period of the study. This includes significant cardiovascular, neurologic, gastrointestinal, endocrine, renal, or psychiatric disorders unrelated to hemophilia identified by key laboratory abnormalities or medical history.
E.15 At screening, anticipated need of surgery during the study or planned surgery scheduled to occur during the study.
E.16 Completion of a surgical procedure within 14 days prior to screening, or currently receiving additional BPA infusion for postoperative hemostasis.
E.17 History of multiple drug allergies or history of allergic reaction to an oligonucleotide or GalNAc.
E.18 Subjects with a central or peripheral indwelling catheter, with a history of venous access complications (such as infections, thrombosis) leading to hospitalization and/or systemic anticoagulation therapy in the last 12 months. (Note: Heparin flushing of a catheter is not to be considered systemic anticoagulation.)
E.19 History of intolerance to SC injection(s).

### Prior/Concomitant Therapy

E.20. Current participation in ITI therapy.
E.21 The use of emicizumab (Hemlibra®) within 6 months prior to screening.

### Prior/Concurrent Clinical Study Experience

E.22 Current or future participation in another clinical study, scheduled to occur during this study, involving an investigational product other than fitusiran or an investigational device; in order to participate in this study, participant must discontinue the investigational product or investigational device at least 30 days (or 5× the investigational product half-life, whichever is longer) prior to dosing (Day 1).

### Study Intervention

Study intervention is defined as any investigational intervention(s), marketed product(s), placebo, or medical device(s) intended to be administered to a study participant according to the study protocol. Fitusiran is administered every 4 weeks, as shown in Table 5 below.

TABLE 5
Overview of fitusiran administered

| | | |
|---|---|---|
| ARM name | Cohort 1 (22 kg | Cohort 2 (8 kg |
| | to <45 kg) | to <22 kg) |
| Fitusiran dose | Solution for injection | Solution for injection |
| formulation | in PBS | in PBS |
| Unit dose strength(s) | 100 mg/mL | 100 mg/mL |
| Starting Dosage level | 10 mg every 4 weeks | 5 mg every 4 weeks |
| Escalation Dosage level | 20 and 30 mg every 4 | 10 and 20 mg every 4 |
| | weeks | weeks |
| De-escalation Dosage | 2.5 and 5 mg every | 1.25 and 2.5 mg every |
| level | 4 weeks | 4 weeks |
| Route of administration | Subcutaneous | Subcutaneous |

Based on the safety and PD data observed on the previous cohort, fitusiran doses may be decreased or increased from those initially planned for the next cohort. The decision to modify the fitusiran doses will be based on the AT activity level; change in the planned dose may occur only after consultation between the DMC and the Sponsor. Following 12 weeks of treatment with starting, escalation or de-escalation prophylactic doses of fitusiran, participants who remain on fitusiran will continue in the study until the completion of treatment periods in all dosing cohorts and the pediatric dosing has been finalized. A dose increase up to the next weight-based dose may be considered on an individual participant basis if a participant's growth results in inclusion in a higher weight group and as clinically indicated per the discretion of the Investigator in consultation with the study Medical Manager.

For the first 7 days of the fitusiran starting dose onset period, participants will continue their pre-study BPA therapies for hemophilia (prophylaxis or on-demand) on a regimen consistent with their pre-study regimen, within the general recommendation of the approved prescribing information, and per Investigator discretion. Bleeding episodes management should be per the local standard practice for episodic use of BPAs and as per Investigator discretion; however, reduced dosing is recommended, where possible. After the first 7 days of fitusiran starting dose, participants will discontinue BPA prophylaxis as applicable, and all participants will treat breakthrough bleeding episodes with on-demand BPA therapy as necessary per the bleeding episode management guidelines in this protocol.

### Concomitant Therapy

Any medication or vaccine (including over-the-counter or prescription medicines, vitamins, and/or herbal supplements) that the participant is receiving at the time of enrollment or receives during the study must be recorded. Local standard treatment of hemophilia is considered to be, but is not limited to, intravenous (IV) infusion of BPAs (e.g., recombinant activated factor VII (rFVIIa; NovoSeven®), and activated prothrombin complex concentrate (aPCC; FEIBAR). Use of these agents is described as follows and details must be captured in the participant's eDiary:
Use of prothrombin complex concentrates for bleeding episode management is not permitted;
Use of emicizumab (Hemlibra®) during the study is not permitted; and
Antifibrinolytics may be used as single agents but may not be used in combination with BPA; use of FEIBA® and NovoSeven® as combination therapy is not recommended.

### AT Activity

Blood samples will be collected for assessment of AT activity levels according to Table 2. On fitusiran dosing days, samples will be collected within 4 hours prior to dosing (pre-dose). Antithrombin levels will be determined by a validated assay. Results will be collected and interpreted by a central laboratory.

Following administration of the final fitusiran dose, AT activity level will be monitored at monthly (or about every 4 weeks) intervals until returning to an activity level of approximately 60% (per the central laboratory) or per Investigator discretion in consultation with the study Medical Manager.

The optimal therapeutic dose for each participant is defined as the dose that renders this participant's steady state AT within the target range of 15-35% and does not meet the criterion for clinical-based escalation. A steady state is considered to have been achieved after the third injection of a fitusiran dose, as modeling and simulations have shown that the majority of patients will be at steady state at this time.

### Management of Bleeding Episodes

The occurrence of bleeding episodes is a typical characteristic of hemophilia; bleeding episodes will be recorded as efficacy assessments of fitusiran and will not be considered as AEs unless the criteria for SAEs are met. Investigators are required to establish and provide instructions for an individualized bleed management plan based on the guidelines in Table 4 for each participant. It is expected that most participants will be entering an OLE study shortly after completing a fitusiran study. However, given that it is possible that participants may have a gap of varying duration between studies, and thus have varying AT activity levels at study entry, approaches to bleed management are described below based on AT activity levels at study entry.

### Bleeding Episode Definitions

A bleeding episode is defined as any occurrence of hemorrhage that requires administration of BPA infusion, e.g., hemarthrosis, muscle, or mucosal bleeding. The definition of bleeding episode types described below is based on consensus opinion of International Society on Thrombosis and Hemostasis (ISTH) as reflected in a recent publication (22).

The start time of a bleeding episode will be considered the time at which symptoms of a bleeding episode first develop. Bleeding or any symptoms of bleeding at the same location that occur within 72 hours of the last injection used to treat a bleeding episode at that location will be considered a part of the original bleeding episode, and will count as 1 bleeding episode towards the ABR. Any bleeding symptoms that begin more than 72 hours after the last injection used to treat a bleeding episode at that location will constitute a new bleeding episode.

A spontaneous bleeding episode is a bleeding episode that occurs for no apparent or known reason, particularly into the joints, muscles, and soft tissues.

A joint bleeding episode is characterized by an unusual sensation in the joint (“aura”) in combination with 1) increasing swelling or warmth over the skin over the joint, 2) increasing pain, or 3) progressive loss of range of motion or difficulty in using the limb as compared with baseline.

A muscle bleed may be characterized by pain, swelling, and loss of movement over the affected muscle group.

A target joint is defined as a joint where three or more spontaneous bleeding episodes in a single joint within a consecutive 6-month period has occurred; where there have been ≤2 bleeding episodes in the joint within a consecutive 12-month period, the joint is no longer considered a target joint.

A traumatic bleeding episode is one that is caused by a known injury or trauma. Bleeding episodes sustained during sports and recreation will be counted as traumatic bleeding episodes.

Bleed Management Guidelines for Participants with AT Activity Level of ≥60% (Per Central Laboratory) at Study Entry

After Day 1 of fitusiran dosing when the AT activity level is ≥60%, it is recommended that participants and/or their caregiver call the Investigator prior to dosing with BPA.

As quickly as 7 days after the initial fitusiran dose, the majority of participants will have AT activity levels at or below 60% activity. By 14 days after the first fitusiran dose, it is expected that most of participants will have AT activity lowering of >50%. Based on these AT activity kinetics, it is recommended that participants continue with their standard BPA regimens for the first week following initiation of fitusiran dosing, with institution of the protocol-specific bleed management guidelines with reduced BPA starting the second week after initiation or re-initiation of fitusiran dosing, as described below and in Table 4.

Bleed Management Guidelines for Week 2 and Beyond, and for Participants with AT Activity Level of <60% (Per Central Laboratory) at Study Entry

When a participant experiences symptoms that may be consistent with bleeding episodes, the following steps should be followed:
1. The participant and/or their caregiver should be instructed to call the study site to discuss symptoms to determine whether or not they are consistent with a bleeding episode and to discuss the appropriate BPA dose to use. This interaction between participant and/or their caregiver and Investigator is recommended prior to the administration of each dose of BPA. Confirmation of bleeding episodes at the study site prior to treatment may be considered.
2. If a determination is made that symptoms require treatment, the recommended treatment algorithm for bleeding episodes is described below:
   a) A single dose can be administered according to the guidelines in Table 4.
   b) The participant and/or their caregiver should be instructed to re-evaluate symptoms in 24 hours for bleeding episodes treated with aPCC and in 2 to 3 hours for bleeding episodes treated with rFVIIa.
3. If a second dose (in the case of aPCC) or a third dose (in the case of rFVIIa) is needed, the participant and/or their caregiver must call the study site before dosing.
   a) Consider evaluation and treatment of the participant at the study site and confirmation of bleeding episodes when any repeat doses are needed.
   b) If more than 2 doses of aPCC or 3 doses of rFVIIa are needed, the participant should be seen at the study site within 48 to 72 hours.
4. Doses should not be administered at less than 24-hour intervals (except rFVIIa as indicated in Table 4).
5. Doses should not exceed the protocol-recommended maximum dose indicated in Table 4.
6. Consultation with the study Medical Manager and Clinical Advisor should be considered for the following clinical circumstances, that may warrant AT replacement:
   a) Doses of BPA higher than those recommended in Table 4.
   b) Dosing of BPA at decreased intervals than those recommended in Table 4.
   c) Multiple or repeat doses of BPA.
7. Antifibrinolytics may not be used in combination with BPA.

### Bleeding Episode Management Following Discontinuation of Fitusiran

Participants who opt to discontinue fitusiran may resume standard prophylaxis or on-demand dosing with BPAs when their AT activity level returns to approximately 60% (per the central laboratory). An earlier restart of standard treatment may be considered in conjunction with consultation from the study Medical Manager, if a strong medical need arises (e.g., increased frequency of bleeding). If full doses of BPA are required to achieve hemostasis prior to full AT recovery (approximately 60% AT activity per the central laboratory), AT replacement should be considered.

### Management of Surgery

The perioperative treatment plan should be developed using the same principles as bleed management described above and the following guidelines:
If the clinical circumstance is such that the recommended doses and/or dose intervals in Table 4 are deemed insufficient for hemostasis, consider AT replacement and manage thrombotic risk as per Investigator practice for a hemophilia participant undergoing that particular surgical procedure.
Nonpharmacologic methods of thromboprophylaxis should also be employed as clinically indicated.

Fitusiran treatment during the perioperative evaluation period is as follows. If the need for a major surgery arises during the study and the procedure is not an emergency or urgent, it is recommended that the procedure be postponed until after completion of the study. For minor operative procedures, dosing with fitusiran may continue uninterrupted.

If the need for emergency or urgent major surgery arises during the study, the participant should be managed medically according to the guidelines above. If a fitusiran dose is scheduled to occur on or in close proximity to the day of surgery, or anytime during the perioperative period, the dose should be withheld. The Perioperative Evaluation Period is defined as the day of the surgery through the final day on which supplemental hemostatic or antithrombotic treatments are administered as part of the perioperative treatment plan. Fitusiran dosing may be resumed at the next scheduled visit following the Perioperative Evaluation Period at the discretion of the Investigator.

Minor surgery is defined as any invasive operative procedure in which only skin, mucous membranes, or superficial connective tissue is manipulated and does not meet the criteria for major surgery (e.g., dental extraction of <3 non-molar teeth). Minor surgical procedures may be performed at a local health care provider institution.

Major surgery is defined as any invasive operative procedure that requires any of the following:
Opening into a major body cavity (e.g., abdomen, thorax, skull).
Operation on a joint,
Removal of an organ,
Dental extraction of any molar teeth or ≥3 non-molar teeth,
Operative alteration of normal anatomy, and
Crossing of a mesenchymal barrier (e.g., pleura, peritoneum, dura).

### Rescue Medicine

The following rescue medications may be used: antithrombin concentrate. Antithrombin reversal should follow labeled product recommendations for the prevention of perioperative thrombosis in participants with AT deficiency, and participant doses individualized to target 80% to 120% AT activity level.

### Dose Modification

Subcutaneous therapy with fitusiran will be used for dosing in each study cohort. Based on the observed safety and PD data, fitusiran doses may be decreased or increased from those initially planned for the next cohort, but will not exceed 30 mg. The decision to modify the fitusiran doses will be based on the AT activity level; change in the planned dose may occur only after consultation between the DMC and the sponsor. Participants who remain on fitusiran will continue in the study until the completion of the treatment periods in all dosing cohorts and the pediatric dose and regimen has been finalized. A dose increase up to the next weight-based dose may be considered if a participant's growth results in inclusion in a higher weight group and as clinically indicated per the discretion of the Investigator in consultation with the study Medical Manager.

### Antithrombin Level Criteria for a Dose Adjustment for Cohort 1

For Cohort 1, participates will start or resume fitusiran at a dose of 10 mg every 4 weeks. As shown in FIG. 3, participants receiving fitusiran at a dose of 10 mg every 4 weeks may be up titrated to an escalation dose of 20 mg every 4 weeks if they have 2 steady state (SS) AT values greater than 35%:
Pre-dose AT values at Week 8 and Week 12 are used for assessment; and
If escalation rules are met, the participant will receive fitusiran 20 mg every 4 weeks starting Week 16.

As shown in FIG. 3, participants receiving the 20 mg escalation dose maybe further up titrated to an escalation dose of 30 mg every 4 weeks if they have 2 steady state AT values greater than 35%:
Pre-dose AT values at Week 24 and Week 28 are used for assessment.
If escalation rules are met, the participant will receive fitusiran 30 mg every 4 weeks, starting Week 32.

In the rare circumstance that participants do not meet criteria for escalation at the above mentioned timepoints and later meet those criteria, the investigator will consult the Medical manager prior to any escalation.

In the unlikely circumstance that a participant has more than 1 AT activity level less than 15% (within a 12-month period) after escalation to 20 mg or 30 mg every 4 weeks, the investigator will consult with the study Medical Manager regarding further treatment with fitusiran.

Participants receiving fitusiran at the starting dose of 10 mg every 4 weeks with more than 1 AT activity level less than 15% on this dose (within a 12-month period) will be allowed to de-escalate to a fitusiran dose of 2.5 mg every 4 weeks. This lower fitusiran dose should be administered only after the participant's AT activity level reaches ≥22%. Participants with more than 1 AT level less than 15% on this lower dose (within a 12-month period) must permanently discontinue fitusiran.

Participants in Cohort 1 who had to discontinue fitusiran prior to this protocol amendment because of AT activity level <15% will be able to resume fitusiran dosing on the lower dose of 2.5 mg, administered every 4 weeks. This lower fitusiran dose should be administered only after the participant's AT activity level reaches ≥22%.

Participants receiving a de-escalated fitusiran dose of 2.5 mg every 4 weeks may be up titrated to a fitusiran dose of 5 mg every 4 weeks if they have 2 steady state AT values greater than 35%.

At each dose level, upon the first AT activity level <15%, the participant must have another AT measurement within 1 week of site receipt of the results. If the result is <15%, this will be considered the second AT activity level <15%. Participants with 1 AT activity level <15% must not receive fitusiran at their current dose regimen until the AT activity level from the second measurement is available to guide management.

### Antithrombin Level Criteria for a Dose Adjustment for Cohort 2

For Cohort 2, participates will start or resume fitusiran at a dose of 5 mg every 4 weeks. As shown in FIG. 4, participants receiving fitusiran at a dose of 5 mg every 4 weeks may be up titrated to an escalation dose of 10 mg every 4 weeks if they have 2 steady state AT values greater than 35%:
Pre-dose AT values at Week 8 and Week 12 are used for assessment.
If escalation rules are met, the participants will receive fitusiran 10 mg every 4 weeks, starting Week 16.

As shown in FIG. 4, participants receiving the 10 mg escalation dose maybe further up titrated to an escalation dose of 20 mg every 4 weeks if they have 2 steady state AT values greater than 35%:
Pre-dose AT values at Week 24 and Week 28 are used for assessment.
If escalation rules are met, the participants will receive fitusiran 20 mg every 4 weeks, starting Week 32.

In the rare circumstance that participants do not meet criteria for escalation at the above mentioned timepoints and later meet those criteria, the investigator will consult the study Medical Manager prior to any escalation.

In the unlikely circumstance that a participant has more than 1 AT activity level less than 15% (within a 12-month period) after escalation to the dose of 10 mg or 20 mg every 4 weeks, the investigator will consult with the study Medical Manager regarding further treatment with fitusiran.

Participants receiving fitusiran at the starting dose of 5 mg every 4 weeks with more than 1 AT activity level less than 15% on this dose (within a 12-month period) will be allowed to de-escalate to a fitusiran dose of 1.25 mg every 4 weeks. This lower fitusiran dose should be administered only after the participant's AT activity level reaches ≥22%. Participants with more than 1 AT level less than 15% on this lower dose (within a 12-month period) must permanently discontinue fitusiran.

Participants receiving a de-escalated fitusiran dose of 1.25 mg every 4 weeks may be up titrated to a fitusiran dose of 2.5 mg every 4 weeks if they have 2 steady state AT values greater than 35%.

At each dose level, upon the first AT activity level <15%, the participant must have another AT measurement within 1 week of site receipt of the results. If the result is <15%, this will be considered the second AT activity level <15%. Participants with 1 AT activity level <15% must not receive fitusiran at their current dose regimen until the AT activity level from the second measurement is available to guide management.

### Clinical Basis Criteria for a Dose Adjustment

The Investigator may request permission from the study Medical Manager to escalate the study participant to a higher dose of fitusiran, despite an AT activity level ≤35% if:
At least 2 doses of fitusiran at the current dose level have been administered, and
The Investigator judges suboptimal bleeding control at the current dose level, defined as 2 or more treated bleeds within a 12-week period starting with the third fitusiran injection at the current dose.

AT activity levels and additional clinical data, as applicable, will be considered in the dose escalation of individual participants.

If the Investigator believes that a specific participant warrants dose escalation based on a different reason, they may discuss the case with the study Medical Manager.

### Assessment of Treatment Response

International Society on Thrombosis and Hemostasis recommendations are provided in Table 6 below for assessment of treatment response.

TABLE 6
Assessment of treatment of acute joint/muscle bleeding episodes

| Category | Response |
|---|---|
| Excellent | Complete pain relief within 8 hours and/or complete |
| | resolution of signs of bleeding after the initial injection and |
| | not requiring any further replacement therapy for relief of |
| | persistent symptoms and signs in the same joint within 72 |
| | hours |
| Good | Significant pain relief and/or improvement in signs of |
| | bleeding within approximately 8 hours after a single injection, |
| | but requiring more than 1 dose of replacement therapy within |
| | 72 hours for complete resolution |
| Moderate | Modest pain relief and/or improvement in signs of bleeding |
| | within approximately 8 hours after the initial injection and |
| | requiring more than 1 injection within 72 hours but without |
| | complete resolution |
| None | No or minimal improvement, or condition worsens, within |
| | approximately 8 hours after the initial injection |

### Example 2: Computational Prediction of AT Levels in Pediatric Patients

The pediatric patient population was segregated into two groups: Cohort 1 (22 kg to <45 kg) and Cohort 2 (8 kg to <22 kg). The dose escalation and de-escalation schemes for Cohorts 1 and 2 are described in Example 1 above.

A target AT window of 15% to 35% was selected to minimize the risk of vascular thrombosis while maintaining efficacy All PK/PD modeling described below was therefore conducted with the objective of finding dosing regimens that could maintain AT activity between 15% and 35%.

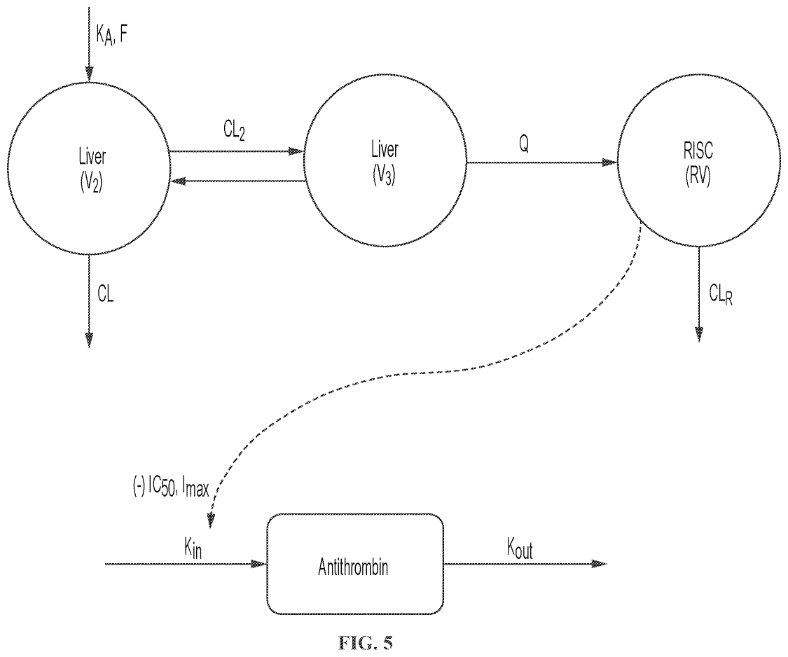

A pharmacokinetic/pharmacodynamic (PK/PD) model as shown in FIG. 5, which illustrates the dynamics of plasma AT activity for patients treated with fitusiran, was developed using AT activity data from 274 participants from adult and adolescent Phase 1, Phase 2 and Phase 3 studies (Phase 3 data includes patients at 80 mg QM and 50 mg Q2M).

The pediatric PK/PD model used to select dosing regimens was based on the adult PK/PD base model that was scaled to pediatric population using bodyweight based allometry. The parameter estimates of the adult PK/PD base model and bodyweight allometric exponents to scale the model to the pediatric population are shown in Table 7.

TABLE 7
Pediatric PK/PD model with allometric
exponents scaled from adult model

| | | | | RSE | |
|---|---|---|---|---|---|
| | | | | (relative | |
| | | Param- | | standard | Allo- |
| Param- | Parameter | eter | Population | error | metric |
| eter | description | unit | estimate | in %) | exponent |
| KA | absorption rate | 1/h | 0.157 | — | — |
| | | | (FIX | | |
| V2 | liver central | g | 3300 | — | 1 |
| | volume | | (FIX) | | |
| V3 | liver peripheral | g | 6075 | — | 1 |
| | volume | | (FIX) | | |
| CL | clearance from | g/h | 39.6 | — | 0.75 |
| | liver central | | (FIX) | | |
| | compartment | | | | |
| CL₂ | liver inter- | g/h | 27.2 | — | 0.75 |
| | compartmental | | (FIX) | | |
| | clearance | | | | |
| Q | clearance | g/h | 0.0195 | — | 1.04 |
| | into RISC | | (FIX) | | |
| | compartment | | | | |
| RV | RISC volume | g | 9375 | — | 1 |
| | | | (FIX) | | |
| CLR | clearance | g/h | 11.31 | 2.14 | 0.72 |
| | from RISC | | | | |
| | compartment | | | | |
| IC50 | RISC | μg/g | 0.0002767 | 6.08 | — |
| | concentration | | | | |
| | for 50% of | | | | |
| | maximum | | | | |
| | effect | | | | |
| Iₘₐₓ | maximum | — | 0.9450 | 0.29 | — |
| | inhibition | | | | |
| | of AT | | | | |
| | production | | | | |
| Kᵢₙ | AT production | %/h | 1.105 | 4.27 | — |
| | rate | | | | |
| Kₒᵤₜ | AT elimination | 1/h | 0.01138 | 4.08 | — |
| | rate | | | | |
| F | Dose effect on | — | −0.279 | — | — |
| | bioavailability | | (FIX) | | |

Inter individual variability (IIV)

| | | | | RSE |
|---|---|---|---|---|
| | | | | (relative |
| | Parameter | Parameter | | standard |
| Parameter | description | unit | % CV | error in %) |
| IIVCLR | IIV on CLR | — | 24.89 | 17.45 |
| IIVIC50 | IIV on IC50 | — | 60.85 | 11.90 |
| IIVKout | IIV on Kout | — | 10.15 | 40.56 |

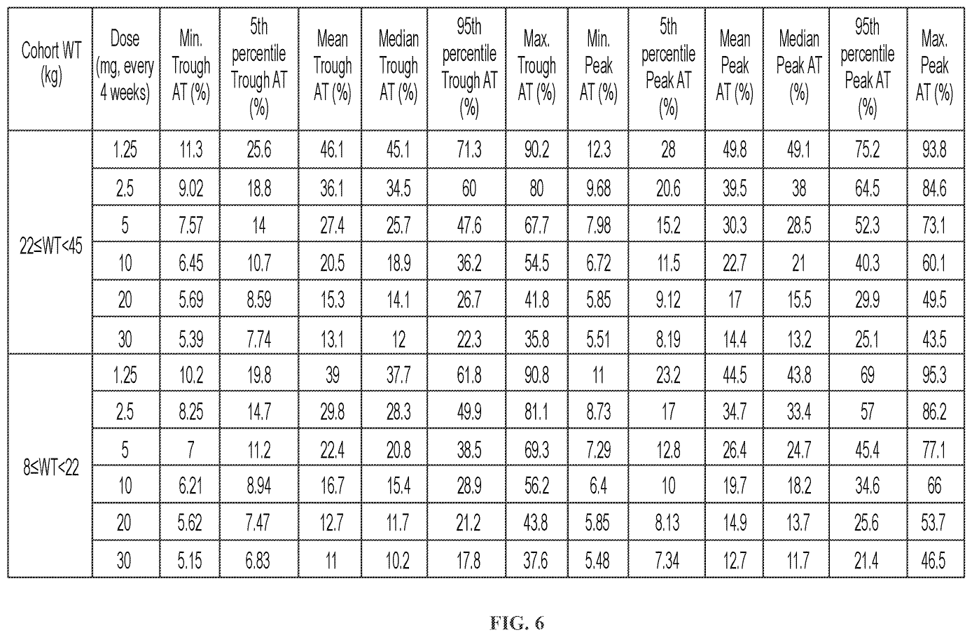

The pediatric PK/PD model with the parameter estimates as shown in Table 7 was used to simulate AT activity at steady state for different dosing scenarios in a virtual population of pediatric patients. The distribution of AT activity for multiple dosing regimens for both pediatric cohorts is shown in FIG. 6. The modeled dosing regimens were 1.25, 2.5, 5, 10, 20, or 30 mg Q4W for Cohort 1 and Cohort 2.

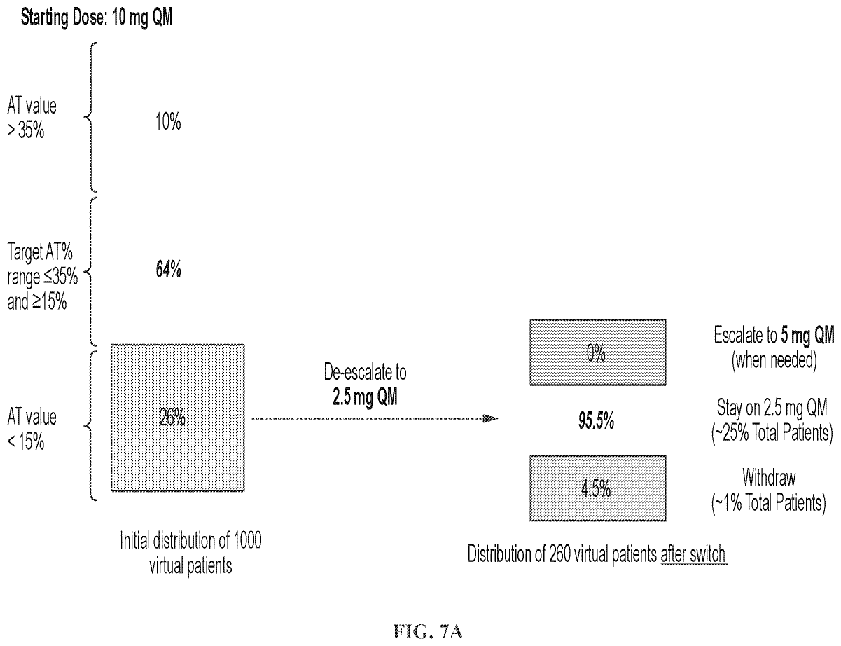

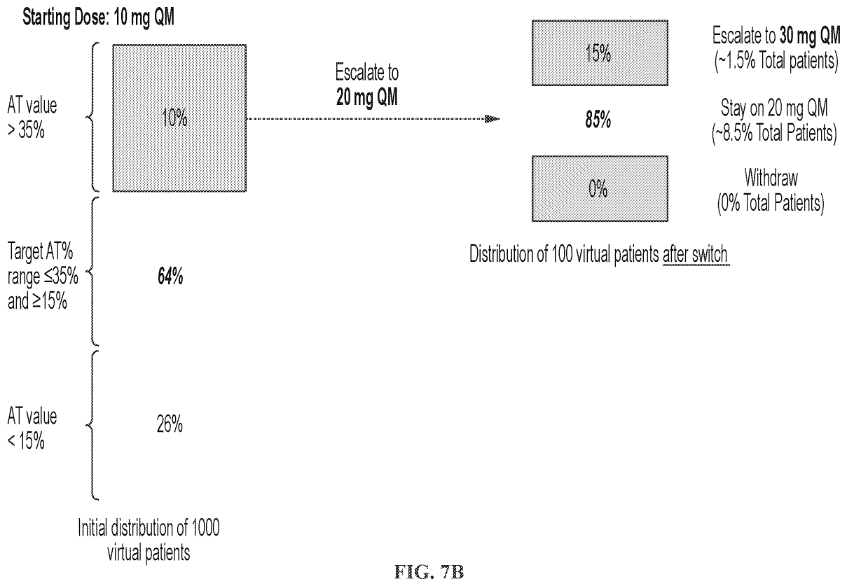

Based on the simulated dosing regimens shown in FIG. 6, for Cohort 1 receiving 10 mg QM fitusiran:
a) about 64% of patients were predicted to have an AT activity level in the 15%≤AT≤35% range;
b) approximately 26% of patients were predicted to have AT activity values <15% and would thus need a lower fitusiran dose for these patients to maintain AT activity within the 15%≤AT≤35% range; a de-escalation dose of 2.5 mg QM for these patients would help 95% of the de-escalated patients to achieve AT activity within the 15%≤AT≤35% range (FIG. 7A); and
c) approximately 10% of patients were predicted to have AT activity values >35% and would thus need a higher dose for these patients to maintain AT activity within the 15%≤AT≤35% range; a dose escalation to 20 mg QM was supported by the pediatric PK/PD model simulations and a further escalation to 30 mg QM was maintained in case it was needed (FIG. 7B).

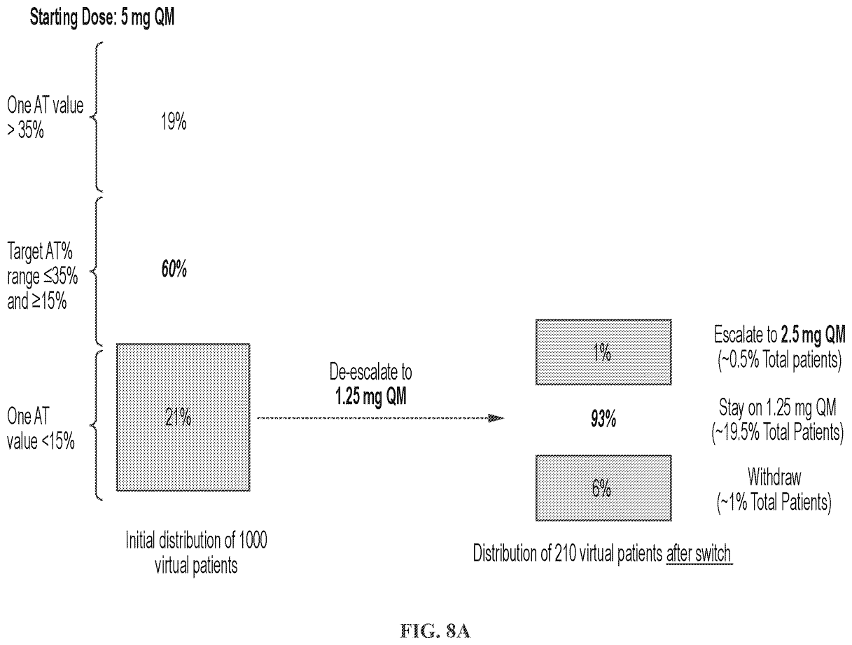

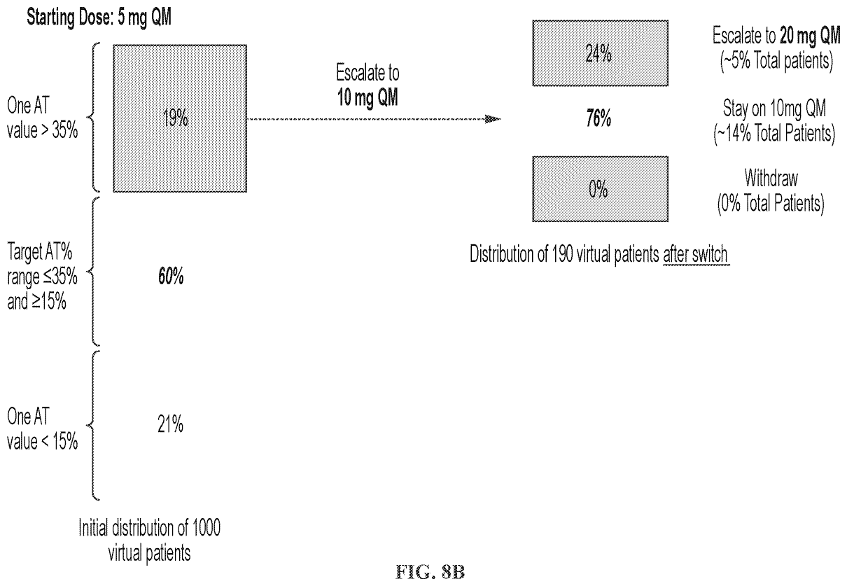

Based on the simulated dosing regimens shown in FIG. 6, for Cohort 2 receiving 5 mg QM fitusiran:
a) approximately 60% of patients were predicted to have AT activity values within the 15%≤AT≤35% range;
b) approximately 21% of patients were predicted to have AT activity values <15% and would thus need a lower dose for these patients to maintain AT activity within the 15%≤AT≤35% range; a de-escalation dose of 1.25 mg QM for these patients then would help 94% of the de-escalated patients to achieve AT activity within the 15%<AT≤35% range (FIG. 8A); and
c) approximately 19% of patients were predicted to have AT activity values >35% and would thus need a higher dose for these patients to maintain AT activity within the 15%<AT≤35% 35% range; a dose escalation to 10 mg QM (for about 76% patients in this subpopulation) was supported by the pediatric PK/PD model simulations and a further escalation to 20 mg QM (for about 24% of the patients in this subpopulation) was maintained in case it was needed (FIG. 8B).

1. A method of prophylactic treatment of hemophilia A or B in a pediatric patient with or without inhibitors, comprising:
(a) subcutaneously administering to the patient in need thereof fitusiran at a starting dose amount at a selected dosing frequency;
(b) obtaining a measurement of an antithrombin (AT) level in the patient; and
(c) performing one of the following steps:
(i) if the AT level is 15-35%, repeating step (a),
(ii) if the AT level is >35%, subcutaneously administering to the patient fitusiran at a higher dose amount at the selected dosing frequency or at the starting dose amount at a higher dosing frequency, or
(iii) if the AT level is <15%, subcutaneously administering to the patient fitusiran at a lower dose amount at the selected dosing frequency or at the starting dose amount at a lower dosing frequency, optionally wherein step (c) (iii) is performed after a pause in fitusiran administration and after the AT level in the patient has returned to ≥15%, optionally ≥22%.

2. A method of reducing thrombotic risk in a pediatric patient receiving fitusiran for prophylactic treatment of hemophilia A or B with or without inhibitors, comprising:
(a) subcutaneously administering to the patient in need thereof fitusiran at a starting dose amount at a selected dosing frequency;
(b) obtaining a measurement of an antithrombin (AT) level in the patient; and
(c) performing one of the following steps:
(i) if the AT level is 15-35%, repeating step (a),
(ii) if the AT level is >35%, subcutaneously administering to the patient fitusiran at a higher dose amount at the selected dosing frequency or at the starting dose amount at a higher dosing frequency, or
(iii) if the AT level is <15%, subcutaneously administering to the patient fitusiran at a lower dose amount at the selected dosing frequency or at the starting dose amount at a lower dosing frequency, optionally wherein step (c) (iii) is performed after a pause in fitusiran administration and after the AT level in the patient has returned to ≥15%, optionally ≥22%.

3. A method of reducing the frequency of bleeding episodes in a pediatric patient with hemophilia A or B with or without inhibitors, comprising:
(a) subcutaneously administering to the patient in need thereof fitusiran at a starting dose amount at a selected dosing frequency;
(b) obtaining a measurement of an antithrombin (AT) level in the patient; and
(c) performing one of the following steps:
(i) if the AT level is 15-35%, repeating step (a),
(ii) if the AT level is >35%, subcutaneously administering to the patient fitusiran at a higher dose amount at the selected dosing frequency or at the starting dose amount at a higher dosing frequency, or
(iii) if the AT level is <15%, subcutaneously administering to the patient fitusiran at a lower dose amount at the selected dosing frequency or at the starting dose amount at a lower dosing frequency, optionally wherein step (c) (iii) is performed after a pause in fitusiran administration and after the AT level in the patient has returned to ≥15%, optionally ≥22%.

4. A method of reducing the annual bleeding rate (ABR), the annual spontaneous bleeding rate (AsBR), and/or the annual joint bleeding rate (AjBR) in a pediatric patient with hemophilia A or B with or without inhibitors, comprising:
(a) subcutaneously administering to the patient in need thereof fitusiran at a starting dose amount at a selected dosing frequency;
(b) obtaining a measurement of an antithrombin (AT) level in the patient; and
(c) performing one of the following steps:
(i) if the AT level is 15-35%, repeating step (a),
(ii) if the AT level is >35%, subcutaneously administering to the patient fitusiran at a higher dose amount at the selected dosing frequency or at the starting dose amount at a higher dosing frequency, or
(iii) if the AT level is <15%, subcutaneously administering to the patient fitusiran at a lower dose amount at the selected dosing frequency or at the starting dose amount at a lower dosing frequency, optionally wherein step (c) (iii) is performed after a pause in fitusiran administration and after the AT level in the patient has returned to >15%, optionally ≥22%.

5. The method of any one of claims 1-4, wherein all of the starting dose amount, the higher dose amount, and the lower dose amount are in the range of 1 mg to 50 mg, optionally in the range of 1 mg to 30 mg, 1 mg to 20 mg, 1 mg to 10 mg, or 1 mg to 5 mg.

6. The method of claim 5, wherein one of the starting dose amount, the higher dose amount, and the lower dose amount is 1.25 mg, 2.5 mg, 5 mg, 7.5 mg, 10 mg, 20 mg, 30 mg, or 50 mg.

7. The method of any one of claims 1-6, wherein
the selected dosing frequency is every two months or every eight weeks and the higher dosing frequency is every month or every four weeks, or
the selected dosing frequency is every month or every four weeks and the lower dosing frequency is every two months or every eight weeks.

8. The method of any one of claims 1-7, wherein the body weight of the patient is 22 kg to <45 kg.

9. The method of claim 8, wherein the starting dose amount of fitusiran is 10 mg.

10. The method of claim 8 or 9, wherein the selected dosing frequency is every month (QM) or every four weeks (Q4W).

11. The method of claim 10, wherein the starting dose amount is 10 mg and the selected dosing frequency is QM or Q4W, wherein
step (c) (ii) is performed if more than one measurement, optionally two measurements, of the AT level at steady state is >35%, or
step (c) (iii) is performed if more than one measurement, optionally two measurements, of the AT level is <15%.

12. The method of claim 11, wherein step (c) (ii) comprises subcutaneously administering to the patient fitusiran at 20 mg QM.

13. The method of claim 12, comprising, after step (c) (ii), subcutaneously administering to the patient fitusiran at 30 mg QM if the AT level at steady state in the patient after step (c) (ii) is >35%, optionally according to two measurements.

14. The method of any one of claims 11-13, comprising, after step (c) (iii):
(A) subcutaneously administering fitusiran to the patient at 2.5 mg QM, optionally after a pause in fitusiran administration and after the AT level in the patient has returned to ≥22%;
(B) if the AT level of the patient after step (A) is
<15%, optionally according to more than measurement, discontinuing or pausing fitusiran treatment,
15-35%, repeating step (A), or
>35%, optionally according to more than one measurement at steady state, subcutaneously administering to the patient fitusiran at 5 mg QM.

15. The method of any one of claims 1-7, wherein the body weight of the patient is 8 kg to <22 kg.

16. The method of claim 15, wherein the starting dose amount of fitusiran is 5 mg.

17. The method of claim 15 or 16, wherein the selected dosing frequency is every month (QM) or every four weeks (Q4W).

18. The method of claim 17, wherein the starting dose amount is 10 mg and the selected dosing frequency is QM or Q4W, wherein
step (c) (ii) is performed if more than one measurement, optionally two measurements, of the AT level at steady state is >35%, or
step (c) (iii) is performed if more than one measurement, optionally two measurements, of the AT level is <15%.

19. The method of claim 18, wherein step (c) (ii) comprises subcutaneously administering to the patient fitusiran at 10 mg QM.

20. The method of claim 19, comprising, after step (c) (ii), subcutaneously administering to the patient fitusiran at 20 mg QM if the AT level at steady state in the patient after step (c) (ii) is >35%, optionally according to two measurements.

21. The method of any one of claims 18-20, comprising, after step (c) (iii):
(A) subcutaneously administering fitusiran to the patient at 1.25 mg QM, optionally after a pause in fitusiran administration and after the AT level in the patient has returned to ≥22%;
(B) if the AT level of the patient after step (A) is
<15%, optionally according to more than measurement, discontinuing or pausing fitusiran treatment,
15-35%, repeating step (A), or
>35%, optionally according to more than one measurement at steady state, subcutaneously administering to the patient fitusiran at 2.5 mg QM.

22. The method of any one of the preceding claims, further comprising subcutaneously administering fitusiran to the patient at a dose amount and a dosing frequency sufficient to maintain the AT level in the patient at 15-35%, optionally comprising subcutaneously administering fitusiran at:
1.25 mg QM or Q4W,
2.5 mg QM or Q4W,
5 mg QM or Q4W,
7.5 mg QM or Q4W,
10 mg QM or Q4W,
20 mg QM or Q4W,
30 mg QM or Q4W, or
50 mg QM or Q4W.

23. The method of any one of the preceding claims, wherein each AT level measurement is obtained after the patient has received at least two doses of fitusiran at a given dose amount.

24. The method of any one of the preceding claims, further comprising:
(d) after step (c), if the patient has an AT level of 15-35% and has at least two doses of fitusiran at the step (c) dose amount and has two or more treated bleeds within a 12-week period starting with the third fitusiran injection at the step (c) dose amount, subcutaneously administering to the patient a higher dose amount of fitusiran than the step (c) dose amount.

25. The method of claim 24, wherein the step (c) dose amount and the step (d) dose amount are:
1.25 mg and 2.5 mg,
2.5 mg and 5 mg,
5 mg and 10 mg,
10 mg and 20 mg, or
20 mg and 30 mg, respectively.

26. The method of any one of the preceding claims, wherein the patient continues a previous non-fitusiran prophylactic treatment for just one week after the first dose of fitusiran, optionally wherein the previous non-fitusiran prophylactic treatment is a replacement factor treatment or a bypassing agent treatment.

27. A method of prophylactic treatment of hemophilia A or B in a patient with or without inhibitors, or reducing the frequency of bleeding episodes, optionally the ABR, the AsBR, or the AjBR, in the patient, comprising subcutaneously administering to the patient in need thereof 1.25 mg of fitusiran every month or every four weeks.

28. A method of prophylactic treatment of hemophilia A or B in a patient with or without inhibitors, or reducing the frequency of bleeding episodes, optionally the ABR, the AsBR, or the AjBR, in the patient, comprising subcutaneously administering to the patient in need thereof 2.5 mg of fitusiran every month or every four weeks.

29. A method of prophylactic treatment of hemophilia A or B in a patient with or without inhibitors, or reducing the frequency of bleeding episodes, optionally the ABR, the AsBR, or the AjBR, in the patient, comprising subcutaneously administering to the patient in need thereof 5 mg of fitusiran every month or every four weeks.

30. A method of prophylactic treatment of hemophilia A or B in a patient with or without inhibitors, or reducing the frequency of bleeding episodes, optionally the ABR, the AsBR, or the AjBR, in the patient, comprising subcutaneously administering to the patient in need thereof 7.5 mg of fitusiran every month or every four weeks.

31. A method of prophylactic treatment of hemophilia A or B in a patient with or without inhibitors, or reducing the frequency of bleeding episodes, optionally the ABR, the AsBR, or the AjBR, in the patient, comprising subcutaneously administering to the patient in need thereof 10 mg of fitusiran every month or every four weeks.

32. A method of prophylactic treatment of hemophilia A or B in a patient with or without inhibitors, or reducing the frequency of bleeding episodes, optionally the ABR, the AsBR, or the AjBR, in the patient, comprising subcutaneously administering to the patient in need thereof 20 mg of fitusiran every month or every four weeks.

33. A method of prophylactic treatment of hemophilia A or B in a patient with or without inhibitors, or reducing the frequency of bleeding episodes, optionally the ABR, the AsBR, or the AjBR, in the patient, comprising subcutaneously administering to the patient in need thereof 30 mg of fitusiran every month or every four weeks.

34. A method of prophylactic treatment of hemophilia A or B in a patient with or without inhibitors, or reducing the frequency of bleeding episodes, optionally the ABR, the AsBR, or the AjBR, in the patient, comprising subcutaneously administering to the patient in need thereof 50 mg of fitusiran every month or every four weeks.

35. The method of any one of the preceding claims, comprising obtaining a measurement of AT level in the patient every four weeks, every eight weeks, every month, every two months, every four months, every six months, or every 12 months.

36. The method of any one of claims 1-35, wherein the patient has hemophilia A or B with inhibitors, further comprising administering an effective amount of a bypassing agent (BPA) to treat a bleeding episode, wherein the effective amount of the BPA is reduced as compared to the recommended effective amount of the BPA.

37. The method of claim 36, wherein the bypassing agent is activated prothrombin complex concentrate (aPCC) and a single dose of aPCC is no more than 50 U/kg and is optionally 30 U/kg, optionally wherein the aPCC administration is repeated, if needed, in no less than 24 hours.

38. The method of claim 36, wherein the bypassing agent is recombinant factor VIIa (rFVIIa) and a single dose of rFVIIa is no more than 45 μg/kg, optionally wherein the rFVIIa administration is repeated, if needed, in no less than two hours.

39. The method of any one of claims 1-35, wherein the patient has hemophilia A or B without inhibitors, comprising administering an effective amount of a replacement factor to treat a bleeding episode, wherein the effective amount of the replacement factor is reduced as compared to the recommended effective amount of the replacement factor.

40. The method of claim 39, wherein the replacement factor is Factor VIII and a single dose of the replacement factor is no more than 20 IU/kg and optionally is 10 IU/kg, optionally wherein the Factor VIII administration is repeated, if need, in no less than 24 hours.

41. The method of claim 39, wherein the replacement factor is Factor IX and a single dose of the replacement factor is 30 IU/kg and optionally is 20 IU/kg, optionally wherein the Factor IX administration is repeated, if need, in no less than 24 hours for standard half-life FIX or in no less than 5-7 days for extended half-life FIX.

42. The method of any one of the preceding claims, wherein fitusiran is provided in a phosphate-buffered saline (PBS) at 1-200 mg/mL, optionally 6.25 mg/mL, 12.5 mg/mL or 100 mg/mL.

43. The method of claim 42, wherein fitusiran is provided in formulation 1 or formulation 2 shown below.

| | Formulation 1 | Formulation 2 |
|---|---|---|
| Components | Conc. [mg/mL] | Conc. [mg/mL] |
| Fitusiran | 12.5 | 100 |
| (Na salt) | (13.25) | (106) |
| NaH₂PO₄*H₂O | 0.388 | 0.0885 |
| Na₂HPO₄*7H₂O | 0.586 | 1.169 |
| NaCl | 8.7 | 4.909 |
| 0.1N NaOH | q.s. | q.s. |
| 0.1M H₃PO₄ | q.s. | q.s. |
| Purified water | Ad 1 mL | Ad 1 mL |

44. Fitusiran for use in a method of any one of claims 1-43.

45. Use of fitusiran for the manufacture of a medicament to treat hemophilia A or B with or without inhibitors in a method of any one of claims 1-43.

46. A pharmaceutical composition comprising fitusiran for use in a method of any one of claims 1-43.

47. An aqueous fitusiran composition comprising:
12.5 mg/mL fitusiran,
0.388 mg/mL NaH₂PO₄*H₂O,
0.586 mg/mL Na₂HPO₄*7H₂O, and
8.7 mg/mL NaCl,
with a pH of about 7.0-7.1.

48. An article of manufacture comprising the composition of claim 47.

49. An article of manufacture for use in a method of any one of claims 1-43, optionally wherein the article of manufacture is a kit.

50. The article of manufacture of claim 48 or 49, wherein the article of manufacture is a container, optionally a vial, containing one or more doses of fitusiran, each dose being 30 mg, 20 mg, 10 mg, 5 mg, 2.5 mg, or 1.25 mg.

51. The article of manufacture of claim 50, wherein the container is a vial containing 2.5 mg of fitusiran.


SEQUENCE LISTING (2 sequences)

SEQ ID NO: 1 - RNA, 21 nt, synthetic construct
  source 1..21: mol_type = other RNA
  modified_base order(1,3,5,7,9..11,13,15,17,19,21): mod_base = OTHER; note = 2-Prime-fluoro ribonucleosides
  modified_base order(2,4,6,8,12,14,16,18,20): mod_base = OTHER; note = 2-Prime-O-methyl ribonucleosides
  modified_base 1..3: mod_base = OTHER; note = Phosphorothioate linkage sodium salt
  modified_base 3..21: mod_base = OTHER; note = Phosphodiester linkage sodium salt
ggttaacaccatttacttcaa

SEQ ID NO: 2 - RNA, 23 nt, synthetic construct
  source 1..23: mol_type = other RNA
  modified_base order(2,4,6,8,10,14,16,18,20): mod_base = OTHER; note = 2-Prime-fluoro ribonucleosides
  modified_base order(1,3,5,7,9,11..13,15,17,19,21..23): mod_base = OTHER; note = 2-Prime-O-methyl ribonucleosides
  modified_base 1..3: mod_base = OTHER; note = Phosphorothioate linkage sodium salt
  modified_base 21..23: mod_base = OTHER; note = Phosphorothioate linkage sodium salt
  modified_base 3..21: mod_base = OTHER; note = Phosphodiester linkage sodium salt
ttgaagtaaatggtgttaaccag